SEQUENCE LISTING (6 sequences)

SEQ ID NO: 1 - DNA, 20 bp, Artificial Sequence
  HSP60 HepG2 F
gaccaaagacgatgccatgc

SEQ ID NO: 2 - DNA, 20 bp, Artificial Sequence
  HSP60 HepG2 R
gcacagccactccatctgaa

SEQ ID NO: 3 - DNA, 20 bp, Artificial Sequence
  HSP60 Rat F
tggagaggtcatcgtcacca

SEQ ID NO: 4 - DNA, 22 bp, Artificial Sequence
  HSP60 Rat R
cacagctactccatctgagagt

SEQ ID NO: 5 - DNA, 20 bp, Artificial Sequence
  HSP70 P.berghei F
aggaatgccaggaggaatgc

SEQ ID NO: 6 - DNA, 20 bp, Artificial Sequence
  HSP70 P.berghei R
agttggtccacttccagctg

(19) United States
(12) Patent Application Publication
(10) Pub. No.: US 2022/0204916 A1
(43) Pub. Date: Jun. 30, 2022

(54) PARASITE PURIFICATION

(71) Applicant: Imperial College Innovations Limited, London (GB)

(72) Inventors: Andrew Blagborough, London (GB); Joshua Blight, London (GB); Arturo Reyes-Sandoval, London (GB); Katarzyna Sala, London (GB); Jacob Baum, London (GB)

(73) Assignee: Imperial College Innovations Limited, London (US)

(21) Appl. No.: 17/609,240

(22) PCT Filed: May 6, 2020
(86) PCT No.: PCT/GB2020/051108

§ 371 Date: Nov. 5, 2021

(30) Foreign Application Priority Data
May 7, 2019 (GB) 1906404.7

(51) Int. Cl. C12N 1/10; A61K 39/018; A61P 33/06

(52) U.S. Cl. CPC C12N 1/10; A61K 2039/522; A61P 33/06; A61K 39/018

(57) ABSTRACT

The invention relates to parasites, and to methods for purifying a metabolically active obligate parasite of vertebrates and arthropods. The invention is especially concerned with methods of purifying Plasmodium and Theileria parasites, such as P. falciparum and T. parva, and highly motile parasite forms thereof, called sporozoites. The invention also relates to obligate parasites of vertebrates and arthropods purified by the methods of the invention and their use thereof.

The present invention relates to parasites, and to methods for purifying a metabolically active obligate parasite of vertebrates and arthropods. The invention is especially concerned with methods of purifying Plasmodium and Theileria parasites, such as P. falciparum and T. parva, and highly motile parasite forms thereof, called sporozoites. The invention also relates to obligate parasites of vertebrates and arthropods purified by the methods of the invention and their use thereof.

Infection by Plasmodium parasites, the causative agents of malaria disease, commences with ejection of parasites from the salivary glands of a feeding female Anopheles mosquito during a blood meal. Upon delivery into the dermis, these highly motile parasite forms, called sporozoites, must migrate to find a blood vessel, following which they are then delivered to the liver, a silent stage of development that precedes establishment of symptomatic blood stage infection and the precursor of the next mosquito transmission cycle(1). Therapies targeting hepatic stages of infection have been of longstanding interest given the low numbers of parasites that successfully establish liver infection(2) (presenting a natural bottleneck in the lifecycle) and the proven ability of irradiated sporozoites that arrest prematurely in hepatocytes(3,4) to offer long-term protection when delivered as a vaccine(5-7). However, since the discovery of a hepatocyte stage of infection in 1948 by Shortt & Garnham(8), and despite subsequent development of in vitro liver cell cultures(9), relatively little is known about the process of sporozoite infection when compared to blood-stages (10,11). This has stagnated discovery of novel therapeutic targets aimed at blocking liver infection and limited vaccine development to core surface proteins of the sporozoite, such as circumsporozoite protein (CSP), a central component in formulations of the current lead-of-field pre-erythrocytic vaccine RTS,S(12). One of the key stumbling blocks to further elucidation of liver stage biology is the difficulty in getting high numbers of Plasmodium infected hepatocytes in vitro using sporozoites isolated by manual salivary gland dissection (SGD)(10,13-16), the current method of choice for isolating parasites from the mosquito. For example, current best estimates suggest infection rates in vitro are generally less than 1% using the murine malaria model Plasmodium berghei (17-20) and less than 2% for human P. falciparum (10,15,21-24) parasites added to in vitro hepatocyte cultures.

Current methods in use involve the direct addition of sporozoites from salivary gland material onto aseptic hepatocyte (primary or hepatoma) cultures. These are isolated by manual dissection 15 to 21 days post-mosquito feeding on infected blood. Originally described in 1964(25) with only minor variations since (9,10,13,15,17-19,26), this method involves mosquito decapitation, gland removal and homogenisation to release sporozoites. Dissection is, however, time-consuming, introduces multiple safety risks, is unstandardized and, requires a skilled technician, who still take >1 hr to dissect 40-60 mosquitoes. Critically, time taken from extraction to addition to culture has been shown as critical for subsequent viability(27), limiting attainable infectious-sporozoite yield, a key requirement for high throughput screening(28). Dissection is also not aseptic and, as such, addition of sporozoites to cultures is commonly associated with bacterial and yeast contamination(22), destroying intra-hepatocytic development. This is especially important for P. falciparum cultures which require 7-10 days to fully develop into hepatic schizonts(29). Furthermore, it is accepted within the field that the use of antifungals and antibiotics to limit contamination(13, 15, 22) can be detrimental for parasite viability. Finally, dissected sporozoites are also frequently contaminated with mosquito-associated material including large debris, protein and lipids(22). Whilst the effect of this debris on hepatocyte infectivity has not been determined, mosquito contaminants have been shown to inhibit sporozoite motility(30) and act as an immune modulator in vivo(31) suggesting their presence markedly influences infection progression.

Attempts at methods of sporozoite isolation aimed at bypassing SGD have included centrifugation through glass wool (32) and compression between glass plates (33). These, however, consistently yielded poor parasite purity that necessitated combination with density gradients (24,34-39). Whilst the addition of gradients increased yields of sporozoites through increased processing capacity, lengthy centrifugation times were required and output was still contaminated with mosquito material (22,40). Other methods have also been developed, including ion exchange chromatography in 1978 by Nawrot et al (41,42), which involved coating sporozoites with BSA prior to injection into a DEAE-cellulose column, and later free-flow electrophoresis (FFE) (40). This latter method involves a liquid form of electrophoresis commonly used to separate organelles under native conditions based on net surface charge (43) and was used by Heidrich et al in 1983 to purify sporozoites. The poor yields or complexity of both methods has, however, limited interest in their usage.

East Coast fever (ECF) is the major cause of livestock loss in sub-Saharan Africa, often referred to as the bovine malaria. The disease is caused by the apicomplexan parasite from the genus Theileria, which are transmitted via the bite of infected ticks. ECF is associated with Theileria parva transmitted from cattle to cattle by the brown ear tick, Rhipicephalus appendiculatus. Sporozoites from the tick secrete into the feeding site of the animal. Sporozoites enter lymphoblasts to form a schizont. There is a clonal expansion of schizonts and then multiply by merging to form merozoites. The merozoites go into erythrocytes and invade the cells and enter into the piroplasm stage. When a tick ingests the piroplasms, the parasites undergo syngamy in the gut and can move to hemolymph of the tick. The motile kinetes can infect the salivary glands. From this, sporogony occurs to create sporozoites to continue the life cycle.

In the sub-Saharan African region, ECF is estimated to kill ˜1 million cattle/year with annual economic losses of ˜USD300 million (73). The current approach to control of ECF is vaccination with drug coverage, referred to as the infection and treatment method (ITM). The current best-practice ITM vaccine is called the Muguga cocktail trivalent vaccine, designed following experiments performed over 40 years ago (74) combining three different Theileria isolates (called Muguga, Serengeti-transformed and Kiambu-5), given as a homogenate with daily oxytetracyclne chemoprophylaxis for 4 days. This provides broad-spectrum immunity to ECF (74). The cost of ITM is relatively high at an estimated US$7 per animal, although the savings made to farmers is considerable, weighed against the cost of treatment or loss of stock (75). Given the high cost and relatively crude nature of the ITM homogenate production, efforts aimed at reducing cost of the vaccine plus increasing efficacy would have major economic impact for African farmers.

The inventors have combined use of an iohexol density gradient or gel filtration with FFE using whole or dissected arthropods in a protocol that is amenable to high throughput applications, providing parasites that can show significantly improved in vitro infectivity and purity compared to manual salivary gland dissection. When used in conjunction with dissected salivary glands as starting material, parasites are also found to be aseptic.

Accordingly, in a first aspect of the invention there is provided a method for purifying a metabolically active obligate parasite of vertebrates and arthropods, the method comprising:
(A) providing an arthropod infected with an obligate parasite of vertebrates and arthropods;
(B) a) (i) homogenising the arthropod of step (A); and
   (ii) filtering the homogenate of step (i); and/or
   (iii) pre-purifying the filtered homogenate resulting from step
(ii); or
b) (i) dissecting the salivary gland of the infected arthropod of step
(A); and
   (ii) homogenising the dissected glands from step (i);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

Thus, in one embodiment, the method comprises purifying a metabolically active obligate parasite of vertebrates and arthropods, the method comprising:
(A) providing an arthropod infected with an obligate parasite of vertebrates and arthropods;
(B) (b) (i) dissecting the salivary gland of the infected arthropod of step (A); and
   (ii) homogenizing the dissected glands from step (i);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods

Step (B) (b) may further comprise:
(iii) filtering the homogenate of step (ii); and/or
(iv) pre-purifying the filtered homogenate resulting from step (iii).

Thus, preferably the method comprises:
(A) providing an arthropod infected with an obligate parasite of vertebrates and arthropods; and
(B) (a) (i) homogenising the arthropod of step (A);
   (ii) filtering the homogenate of step (i); and
   (iii) pre-purifying the filtered homogenate resulting from step (ii);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

The method of the invention that utilises filtering and/or pre-purification steps advantageously produces obligate parasites of vertebrates and arthropods that are cleaner and more infective than standard methods in the art. Furthermore, utilising the method of step B (b) advantageously enables the production of obligate parasites of vertebrates and arthropods in a much shorter period of time, i.e. higher throughput, than prior art methods whilst maintaining sufficient purity.

The arthropod that is provided in step (A) may be provided as a whole, or in part.

The skilled person would understand that an obligate parasite of vertebrates and arthropods relates to a parasite that infects both arthropods and vertebrate hosts and requires both hosts to complete its life cycle. The obligate parasite may be an apicomplexan parasite. The obligate parasite may be a parasite selected from the group consisting of a Plasmodium species parasite, a Trypanosoma species parasite, a Leishmania species parasite, a Theileria species parasite and a protozoan parasite of arthropods and vector borne viruses (also called arboviruses). Preferably, the obligate parasite is a Plasmodium species parasite and/or a Theileria species parasite. The obligate parasite may be a species of vector-borne apicomplexan parasite. For example, the obligate parasite may be of the genera Theileria, such as T. parva, T. annulata or T. lestoquardi) or Babesia, such as B. divergens, B. bigemina, B. bovis, or B. major. However, preferably the obligate parasite is a Plasmodium species parasite, more preferably P. falciparum, P. vivax, P. ovale, P. malariae or P. knowlesi and most preferably P. falciparum.

The inventors have applied their method to the purification of a related apicomplexan T. parva, in infected ticks, showing production of pure Theileria sporozoites that can advantageously be used directly in ITM vaccination, showing the broad applicability of the inventor's method in purifying obligate parasites of vertebrates and arthropods. Thus, in one embodiment the obligate parasite may be a Theileria species parasite. Preferably, the Theileria species parasite may be selected from T. parva, T. annulata or T. lestoquardi. More preferably, the Theileria species parasite is Theileria annulata. Most preferably, the Theileria species parasite is Theileria parva.

Preferably, when the parasite is a Theileria species parasite the parasite is at the sporozoite life cycle stage. However, the parasite may be isolated at other life cycle stages.

The Theileria sporozoite stage develops within the tick salivary gland and is transmitted to the bovine host when the tick, e.g. Rhipicephalus appendiculatus, feeds. The sporozoite enters the blood stream and invades a subpopulation of bovine T and B lymphocytes where it differentiates into the multinucleate schizont stage, and in the process induces a rapid clonal expansion of parasitized cells. Schizonts undergo a process of merogony to produce merozoites, which are released by host cell rupture. Merozoites then invade erythrocytes, where they develop into a piroplasm stage. The piroplasm stage is infective to ticks and differentiates into gamete stages in the tick gut. Macro- and micro-gametes fuse to form a zygotes that enter cells of the tick gut epithelium and develop into motile kinetes, which are released into the tick hemocel. Sporogony occurs in the salivary gland cells of the tick although maturation of the parasite sporoblast starts after tick attachment to the host, which results in sporozoites being released into tick saliva.

Accordingly, the other stages of the lifecycle at which the Theileria species parasite may be isolated includes the post-fertilisation zygote. Further life cycles stages at which the Theileria species parasite may be isolated include: merozoite, gametocyte (both male and female), microgamete and the macro gamete stage.

When the obligate parasite is a Plasmodium species parasite, the parasite is preferably at the sporozoite life cycle stage, a stage found in both vertebrate and invertebrate host. However, the parasite may be isolated at other life cycle stages. Other stages of the vertebrate lifecycle stage from which the parasite may be isolated includes the post-fertilization zygote, which the skilled person would understand may be referred to as the ookinete, which infects the mosquito midgut leading to sporozoite formation. Further life cycles stages at which the Plasmodium species parasite may be isolated include: merozoite, gametocyte (both male and female), microgamete and the macro gamete stage. Thus, preferably the obligate parasite is isolated at the life cycle stage selected from: sporozoite, post-fertilization zygote merozoite, gametocyte (both male and female), microgamete and the macro gamete stage. Most preferably, the obligate parasite is isolated at the sporozoite life cycle stage.

The arthropod may be an insect and/or an arachnid. The arthropod may be selected from the group consisting of: a mosquito, Tsetse fly, Rhipicephalus spp. and triatominae spp.

In one preferred embodiment, the arthropod is an insect. Preferably, the insect is a mosquito. The mosquito may be of the subfamily Anophelinae. Preferably, the mosquito is selected from the group consisting of: Anopheles gambiae; Anopheles coluzzi; Anopheles merus; Anopheles arabiensis; Anopheles quadriannulatus; Anopheles stephensi; Anopheles dirus; Anopheles arabiensis; Anopheles funestus; and Anopheles melas.

Most preferably, the mosquito is Anopheles stephensi or Anopheles gambiae.

In another preferred embodiment the arthropod is an arachnid. Preferably, the arachnid is a tick. More preferably, the arachnid is a Rhipicephalus spp. Preferably, the Rhipicephalus spp is Rhipicephalus appendiculatus. Preferably, the obligate parasite is a Theileria species parasite and the arthropod is a Rhipicephalus spp. More preferably, the obligate parasite is Theileria parva and the arthropod is Rhipicephalus appendiculatus.

The vertebrate may be a mammal. Preferably, the mammal is rodent, primate, human bovine, ovine and/or caprine. Preferably, when the attenuated obligate parasite is a Plasmodium spp, the vertebrate is human, primate or rodent. More preferably, when the obligate parasite is a Plasmodium species parasite, the vertebrate is human. Preferably, when the obligate parasite is a Theileria species parasite, the vertebrate is of the family Bovidae, for example bovine, ovine and/or caprine. Preferably, when the obligate parasite is a Theileria species parasite, the vertebrate is bovine. Preferably, when the attenuated obligate parasite is a Theileria species the vertebrate may be a cow, buffalo, sheep or goat. Preferably, when the attenuated obligate parasite is T. parva and T. annulata the vertebrate is a cow or buffalo. Preferably, when the attenuated obligate parasite is T. lestoguardi the vertebrate is a sheep or goat.

When the obligate parasite is a Plasmodium species parasite and the arthropod a mosquito, step (A) may further comprise removing the abdomen of the mosquito prior to homogenization, enabling differentiation of midgut, haemocoel and salivary gland forms of the sporozoite.

The homogenising step may be performed in the presence of a buffer to maintain viability of the obligate parasite. The buffer may be a mammalian buffer, an arthropod buffer, an arachnid buffer or an insect buffer. Preferably the buffer is an insect buffer, more preferably Schneider's Drosophila medium. Preferably the buffer further comprises Fetal Bovine Serum (FBS). The buffer may be present at between 0.1 and 10.0 ml per 100 insects, preferably between 0.5 ml and 5 ml per 100 insects and most preferably between 1 ml and 3 ml per 100 insects. The buffer may be present at 0.5, 1.0, 1.5, 2.0, 2.5, 5.0, or 10.0 ml per 100 insects. Preferably, the buffer is present at 2.0 ml per 100 insects. The buffer may be present at between 1 and 10 ml per 100 arachnids. Preferably, the buffer may be present at between 3 ml and 10 ml per 100 arachnids

Homogenisation may be performed by macerating the arthropod of step (A) in buffer, optionally using borosilicate beads or blending.

Homogenisation steps B(a) (i) or B(b) (ii) may further comprise:
(aa) centrifuging the homogenate at a speed of less than 150 xg, 100 xg, 50 xg, or 25 xg, preferably at a speed of equal to or less than 140 xg, and most preferably at a speed of less than 15 xg, and
(bb) obtaining the supernatant resulting from step (aa).

Preferably, when the arthropod is a Plasmodium species, homogenisation steps B(a) (i) or B(b) (ii) may further comprise:
(aa) centrifuging the homogenate at a speed of less than 150 xg, 100 xg, 50 xg, or 25 xg, preferably at a speed of equal to or less than 140 xg, and most preferably at a speed of less than 15 xg, and
(bb) obtaining the supernatant resulting from step (aa).

Preferably, when the arthropod is a Theileria species, homogenisation steps B(a) (i) or B(b) (ii) may further comprise:
(aa) centrifuging the homogenate at a speed of less than 150 xg, 100 xg, 50 xg, or 25 xg, most preferably at a speed of equal to or less than 100 oxg.
(bb) obtaining the supernatant resulting from step (aa).

Filtering steps may comprise passing the homogenate sequentially through size exclusion filters. The homogenate may be passed through:
(aa) a 200 μm to 70 μm pore size filter;
(bb) a 7 μm to 4 μm pore size filter
(cc) a 4 μm to 25 μm pore size filter;
(dd) a 25 μm to 15 μm pore size filter; and/or
(ee) a 15 μm to 5 μm pore size filter.

Preferably, the homogenate is passed through:
(aa) a 200 μm to 70 μm pore size filter;
(bb) a 7 μm to 4 μm pore size filter
(cc) a 4 μm to 25 μm pore size filter; and
(dd) a 25 μm to 15 μm pore size filter.

The homogenate may be further passed though: (ee) a 15 μm to 5 μm pore size filter.

Most preferably, the homogenate is passed through:
(aa) a 100 μm pore size filter;
(bb) a 70 μm pore size filter
(cc) a 4 μm pore size filter; and
(dd) a 20 μm pore size filter.

The filtration step may be performed by vacuum based filtration that advantageously enables a smaller filter pore size to be used. This involves the creation of a lower pressure below the filter by use of a syringe, vacuum pump or similar apparatus, causing liquid to flow through the filter membrane from high pressure to lower pressure.

Accordingly, the filtering step preferably comprises passing the homogenate sequentially through:
(aa) a 100 μm pore size filter;
(bb) a 70 μm pore size filter
(cc) a 40 μm pore size filter;
(dd) a 20 μm pore size filter; and
(ee) a 10 μm pore size filter.

Pre-purification may be performed by either density gradient purification or gel filtration. Such methods would be known to those skilled in the art.

In one embodiment the pre-purification step is performed by density gradient purification. Preferably density gradient purification is performed using an iohexol density gradient. Pre-purification may be performed with an iohexol density gradient at a temperature of less than 20° C., 15° C., 10° C. or 5° C. Preferably, pre-purification is performed with a iohexol density gradient at 4° c.

The density gradient may comprise between 6% and 25% w/v iohexol. The density gradient may comprise between 6% and 20% w/v iohexol The density gradient may comprise between 10% and 25% w/v iohexol, preferably between 15% and 20% w/v iohexol. The density gradient may comprise 10%, 11%, 12%, 13%, 14%, 15%, 16%, 17%, 18%, 19%, 20%, 21%, 22%, 23%, 24% or 25 w/v iohexol. Preferably, the density gradient comprises 17% w/v iohexol.

The pre-purification step may comprise
(aa) loading the homogenate onto a density gradient;
(bb) centrifuging the homogenate present in the density gradient of step aa); and
(cc) obtaining a parasite enriched fraction from a parasite enriched boundary.

The pre-purification step may further comprise:
(dd) centrifuging the parasite enriched fraction obtained in step (cc);
(ee) aspirating the supernatant resulting from step (dd); and
(ff) resuspending the resulting pellet comprising the parasite in buffer.

The buffer may be the same as that used in the filtration step.

Preferably, centrifugation step bb) may comprise centrifuging at between 1000 xg-5000 xg. Preferably centrifugation step bb) may comprise centrifuging at between 2000 xg and 3000 xg. Most preferably, centrifugation step bb) may comprise centrifuging at about 2400 xg.

Advantageously, the pre-purification step may be performed by gel filtration, which reduces the purification time and is less toxic.

Thus, in another embodiment, the pre-purification step is performed by gel filtration, more preferably by cross-linked dextran (Sephadex®) gel filtration, more preferably G-15 Medium grade Sephadex®) gel filtration.

Preferably, when the pre-purification step is performed by Sephadex® gel filtration, pre-purification step may comprise:
(aa) loading the homogenate onto hydrated Sephadex® columns; and
(bb) eluting the homogenate to obtain a parasite enriched faction.

The pre-purification step may further comprise:
(cc) centrifuging the parasite enriched fraction obtained in step (bb);
(dd) aspirating the supernatant resulting from step (dd); and
(ee) resuspending the resulting pellet comprising the parasite in buffer.

Preferably the columns are pre-equilibrated in same buffer used in the filtration step, preferably the column is between 1.5 and 5 cm in height, most preferably 3 cm height.

The loaded column may be eluted by gravity or pressure (preferably by use of a syringe or or by centrifugation). In one embodiment, the column is eluted by centrifugation, preferably centrifugation is performed at between 150 xg and 6000 xg, for between 30 sec and 5 minutes. Most preferably, centrifugation is performed for 30 seconds at 174 xg.

Separation step (C) may comprise continuous zone electrophoresis or interval zone electrophoresis. Such techniques would be known to those skilled in the art.

Continuous electrophoresis may be performed using the following parameters: FFE machine setup: between 0.4 and 0.5 mm spacer, horizontal chamber, continuous sample injection.

Suitable buffers used for electrophoresis would be well known to those skilled in art. In one embodiment, the buffers used for electrophoresis may be: Separation/counterflow buffer: 10 mM TEA, 10 mM Ac, 250 mM Sucrose pH7.4; Stabilisation buffer: 100 mM TEA, 100 mM Ac, 250 mM Sucrose, pH 7.4.

The running setting may have a voltage of between 650V and 900V, a maximum current of between 150 mA and 250 mA, a maximum wattage of between 150 and 250 W, and a temperature of between 4° C. and 12° C. Continuous Sample injection may be set at up to between 500-2000 μl of sample/hr.

Preferably, continuous electrophoresis is performed using the following parameters:

FFE machine setup utilising a 0.5 mm spacer, a horizontal chamber, and continuous sample injection.

The buffers used for electrophoresis: separation/counterflow buffer may be: 10 mM TEA, 10 mM Ac, 250 mM Sucrose pH7.4; stabilisation buffer: 100 mM TEA, 100 mM Ac, 250 mM Sucrose, pH 7.4.

The running settings of 900V, max 250 mA, max 200 W, a temperature of 10° C. Continuous Sample injection at 1200 uL of sample/hr.

Preferably, electrophoresis is performed using interval zone electrophoresis.

In embodiment, when interval electrophoresis is used, the electrophoresis chamber may be coated with hydroxypropyl methylcellulose (HPMC).

In one embodiment, when interval electrophoresis is used, arthropods may be injected at a concentration of between 50 and 250 arthropods/ml, preferably about 150 arthropods/mL injection.

In one embodiment, the parameters used for interval electrophoresis are as set out in Table 1.

In another embodiment, the parameters for interval electrophoresis used are as follows: FFE Machine Setup: 0.2 mm spacer, horizontal setup, interval flow.

Suitable buffers used for electrophoresis would be well known to those skilled in art. In 20 one embodiment, the buffers are: separation/counterflow buffer: 10 mM TEA, 10 mM Ac, 250 mM Sucrose pH7.4; stabilisation buffer: 100 mM TEA, 100 mM Ac, 250 mM Sucrose, pH 7.4.

Running settings: between 1000V and 1300V, between 150 mA and 250 mA, between 150 W and 200 W, a temperature of between 4° C. and 10° C. Interval sample injection between 500 and 2000 ul of sample/hr.

Preferably, the parameters for interval electrophoresis are as follows: 20 FFE Machine Setup: 0.2 mm spacer, horizontal setup, interval flow.

Buffers: separation/counterflow buffer: 10 mM TEA, 10 mM Ac, 250 mM Sucrose pH7.4; Stabilisation buffer: 100 mM TEA, 100 mM Ac, 250 mM Sucrose, pH 7.4.

Running settings: 1200V, max 250 mA, max 200 W, 10° C., 220 mL/hr and 20 mL/hr.

Interval sample injection at 1600 uL of sample/hr.

In another embodiment, the parameters for interval zone electrophoresis may be as follows: FFE Machine Setup: 0.2 mm spacer, horizontal setup, interval flow.

Buffers: separation/counterflow buffer: 30 mM NaCl, 40 mM BISTRIS, 20 mM EPPS, 170 mM Sucrose, 10 mM Glucose, pH 7.4; Stabilisation buffer (Anode): 150 mM Na2SO4, 40 mM BISTRIS, 20 mM EPPS, pH 7.4; Stabilisation buffer (Cathode): 300 mM NaCl, 40 mM BISTRIS, 20 mM EPPS, 75 mM Sucrose, pH 7.4; Electrode buffer (Anode): 200 mM Na-acetate; Electrode (Cathode): 100 mM NaCl, 100 mM HCl, 200 mM Imidazol.

Running settings: between 350V and 450V, max 250 mA, max 200 W, a temperature of between 4° C. and 10° C., 220 mL/hr and 20 mL/hr. Interval sample injection up to 2000 uL/hr

Preferably, the parameters for interval electrophoresis are as follows:

FFE Machine Setup: 0.2 mm spacer, horizontal setup, interval flow.

Buffers: Seperation/counterflow buffer: 30 mM NaCl, 40 mM BISTRIS, 20 mM EPPS, 170 mM Sucrose, 10 mM Glucose, pH 7.4; Stabilisation buffer (Anode): 150 mM Na₂SO₄, 40 mM BISTRIS, 20 mM EPPS, pH 7.4; Stabilisation buffer (Cathode): 300 mM NaCl, 40 mM BISTRIS, 20 mM EPPS, 75 mM Sucrose, pH 7.4; Electrode (Anode): 200 mM Na-acetate; Electrode buffer (Cathode): 100 mM NaCl, 100 mM HCl, 200 mM Imidazol.

Running settings: 420V, max 250 mA, max 200 W, 10° C., 220 mL/hr and 20 mL/hr.

Interval sample injection at 1600 uL/hr.

Preferably, the fraction comprising the obligate parasite of vertebrates and arthropods obtained in step D is fraction number 13, 14, 15, 16, 17, 18 and/or 19 of the fractions of the sample separated by electrophoresis.

In a preferred embodiment, the method comprises:
(A) providing an arthropod infected with an obligate parasite of vertebrates and arthropods; and
(B) (a) (i) homogenising the arthropod of step (A);
   (ii) filtering the homogenate of step (i);
   (iii) centrifuging the homogenate at a speed of less than 150 xg, 100 xg, 50 xg, or 25 xg, preferably at a speed of equal to or less than 140 xg, and most preferably at a speed of less than 15 xg;
   (iv) obtaining the supernatant resulting from step (iii);
   (v) pre-purifying the filtered homogenate resulting from step (iv) by
      aa) loading the homogenate onto a density gradient;
      (bb) centrifuging the homogenate present in the density gradient of step aa); and
      (cc) obtaining a parasite enriched fraction from a parasite enriched boundary;
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

In a preferred embodiment, the method comprises:
(A) providing an arthropod infected with an obligate parasite of vertebrates and arthropods; and
(B) (a) (i) homogenising the arthropod of step (A);
   (ii) filtering the homogenate of step (i) by passing the homogenate sequentially through size exclusion filters;
   (iii) centrifuging the homogenate at a speed of less than 150 xg, 100 xg, 50 xg, or 25 xg, preferably at a speed of equal to or less than 140 xg, and most preferably at a speed of less than 15 xg;
   (iv) obtaining the supernatant resulting from step (iii);
   (v) pre-purifying the filtered homogenate resulting from step (iv) by
      (aa) loading the homogenate onto a density gradient;
      (bb) centrifuging the homogenate present in the density gradient of step aa);
      (cc) obtaining a parasite enriched fraction from a parasite enriched boundary;
      (dd) centrifuging the parasite enriched fraction obtained in step (cc);
      (ee) aspirating the supernatant resulting from step (dd); and
      (ff) resuspending the resulting pellet comprising the parasite in buffer;
(C) separating the homogenate resulting from step (B) into fractions by continuous zone electrophoresis or interval zone electrophoresis; and
(D) obtaining a fraction from step (C) comprising the obligate parasite of vertebrates and arthropods, thereby obtaining a purified metabolically active obligate parasite of vertebrates and arthropods.

In another preferred embodiment, the method comprises:
(A) providing an arthropod infected with an obligate parasite of vertebrates and arthropods; and
(B) (a) (i) homogenising the arthropod of step (A);
   (ii) filtering the homogenate of step (i);
   (iii) centrifuging the homogenate at a speed of less than 150 xg, 100 xg, 50 xg, or 25 xg, preferably at a speed of equal to or less than 140 xg, and most preferably at a speed of less than 15 xg;
   (iv) obtaining the supernatant resulting from step (iii);
   (v) pre-purifying the filtered homogenate resulting from step (iv) by
      (aa) loading the homogenate onto hydrated Sephadex® columns; and
      (bb) eluting the homogenate to obtain a parasite enriched faction;
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

In another preferred embodiment, the method comprises:
(A) providing an arthropod infected with an obligate parasite of vertebrates and arthropods; and
(B) (a) (i) homogenising the arthropod of step (A);
   (ii) filtering the homogenate of step (i) by passing the homogenate sequentially through size exclusion filters;
   (iii) centrifuging the homogenate at a speed of less than 150 xg, 100 xg, 50 xg, or 25 xg, preferably at a speed of equal to or less than 140 xg, and most preferably at a speed of less than 15 xg;
   (iv) obtaining the supernatant resulting from step (iii);
   (v) pre-purifying the filtered homogenate resulting from step (iv) by
      aa) loading the homogenate onto hydrated Sephadex® columns;
      bb) eluting the homogenate to obtain a parasite enriched faction;
      (cc) centrifuging the parasite enriched fraction obtained in step (bb);
      (dd) aspirating the supernatant resulting from step (dd); and
      (ee) resuspending the resulting pellet comprising the parasite in buffer;
(C) separating the homogenate resulting from step (B) into fractions by continuous zone electrophoresis or interval zone electrophoresis; and
(D) obtaining a fraction from step (C) comprising the obligate parasite of vertebrates and arthropods, thereby obtaining a purified metabolically active obligate parasite of vertebrates and arthropods.

In a second aspect of the invention there is provided an obligate parasite of vertebrates and arthropods obtained or obtainable by the method of the first aspect.

Preferably, the obligate parasite, vertebrate and arthropod is as defined in the first aspect.

The obligate parasite may be live or attenuated. Preferably, the obligate parasite is attenuated such that infection of a host fails to cause disease pathology.

Preferably, when the obligate parasite is a Theileria species, the parasite may be either live or attenuated. Preferably, when the obligate parasite is a Plasmodium species, the parasite may be attenuated.

Advantageously, the inventors have shown that parasites purified using the methods described herein display particularly advantageous properties. For example, the parasites may display an increase in fitness (i.e. their ability to establish liver stage and then blood-stage infection), which is clear at lower infective doses. The parasites may also induce a more robust immune response.

Thus, in a third aspect of the invention there is provided a preparation of live or attenuated obligate parasite of vertebrates and arthropods, wherein:
i) the preparation is capable of inducing a cellular and humoral immune response when introduced into a subject;
ii) the preparation does not comprise detectable amounts of free circumsporozoite protein (CSP);
iii) the preparation does not comprise detectable amounts of arthropod protein, preferably when assessed by silver stain;
iv) the preparation does not comprise detectable bacterial contamination, preferably when assessed by colony forming units;
v) the preparation enables detection of parasite proteins by mass spectrometry; vi) the preparation of parasites show no reduction in gliding motility;
vii) the preparation, when introduced into an in vitro culture of hepatocytes, results in infection of at least 0.5, 1, 2, 3, 4, 5, 6, 7, 8, 9 or 10% of total hepatocytes present in the culture;
viii) the preparation, when introduced into an in vitro culture of peripheral blood mononuclear cells, results in infection of at least 0.5, 1, 2, 3, 4, 5, 6, 7, 8, 9 or 10% of total peripheral blood mononuclear cells present in the culture;
ix) the preparation, when injected in vivo into rodents, shows improved time to patency; and/or
x) the preparation, when injected in vivo into bovines, shows improved time to patency.

Preferably, the obligate parasite of vertebrates and arthropods is as defined in the first aspect. Preferably, the arthropod is as defined in the first aspect.

The obligate parasite may be live or attenuated. Preferably, the obligate parasite is attenuated such that infection of a host fails to cause disease pathology.

Preferably, when the obligate parasite is a Theileria species, the parasite may be either live or attenuated. Preferably, when the obligate parasite is a Plasmodium species, the parasite may be attenuated.

Preferably, the preparation of step vii) is of attenuated obligate parasite of vertebrates and insects. Preferably, a preparation of attenuated Plasmodium species.

Preferably, the preparation of step viii) is of a live or attenuated obligate parasite of vertebrates and arachnids. Preferably, a preparation of live or attenuated Theileria species.

Preferably, the preparation of step ix) is of attenuated obligate parasite of vertebrates and insects. Preferably, a preparation of attenuated Plasmodium species.

Preferably, the preparation of step x) is of a live or attenuated obligate parasite of vertebrates and arachnids. Preferably, a preparation of live or attenuated Theileria species.

The skilled person would understand that gliding motility is a known viability marker, the method of which is well known in the art. This method may involve assessing motility based in the following variables when settles on flat surfaces (assessed by microscopy): Attachment, waving, gliding.

Detection of total arthropod protein may be determined by silver staining, and undetectable amounts may relate to less than 0.25 ng of arthropod protein, preferably insect protein. The method of silver staining is well known in the art.

Detection of CSP parasite protein may be determined by western blotting.

Detection of bacteria may be determined by growth on blood agar places, and undetectable levels may relate to no visible bacterial colonies.

The in vitro culture of hepatocytes may be a culture of human hepatoma cell lines, rodent hepatoma cells lines, primary human hepatocytes, primary rodent hepatocytes, primary bovine hepatocytes or hepatocytes derived from pluripotent stem cells or fibroblasts. The preparation may result in infection of at least 0.5, 1, 2, 3, 4, 5, 6, 7, 8, 9 or 10% of total hepatocytes present in the culture. Preferably, the preparation may result in infection of at least 4% of total hepatocytes present in the culture.

The in vitro culture of peripheral blood mononuclear cells may be a culture of bovine peripheral blood mononuclear cell lines, rodent peripheral blood mononuclear cell lines, primary human peripheral blood mononuclear cell, primary rodent peripheral blood mononuclear cell, or primary bovine peripheral blood mononuclear cell. The preparation may result in infection of at least 0.5, 1, 2, 3, 4, 5, 6, 7, 8, 9 or 10% of total peripheral blood mononuclear cells present in the culture. Preferably, the preparation may result in infection of at least 4% of total peripheral blood mononuclear cells present in the culture.

Preferably, the peripheral blood mononuclear cell is a lymphocyte.

The skilled person would understand that a “cellular immune response” relates to the activation of T-cells and a “humoral immune response” to the activation of B-cells, the processes of which would be known to those skilled in the art.

In a fourth aspect of the invention, there is provided a pharmaceutical composition comprising an attenuated obligate parasite of vertebrates and arthropods of the second or third aspect and a pharmaceutically acceptable vehicle.

Preferably, the obligate parasite of vertebrates and arthropods is as defined in the first aspect.

In a fifth aspect of the invention there is provided a method of preparing the pharmaceutical composition according to the fourth aspect, the method comprising contacting the attenuated obligate parasite of vertebrates and arthropods of the second or third aspect with a pharmaceutically acceptable vehicle.

In a sixth aspect of the invention, there is provided a method of vaccinating a subject, the method comprising administering, or having administered, to a subject in need thereof, a therapeutic amount of an attenuated obligate parasite of vertebrates and arthropods of the second or third aspect, or a pharmaceutical composition of the fourth aspect.

Preferably, the subject is a mammal. The mammal may be a rodent, primate, human, bovine, ovine and/or caprine.

Preferably, when the attenuated obligate parasite is a Plasmodium spp, the subject is human, primate or rodent. More preferably, when the attenuated obligate parasite is a Plasmodium, the subject is human.

Preferably, when the attenuated obligate parasite is a Theileria species the subject is of the family Bovidae, for example bovine ovine and/or caprine. Preferably, when the attenuated obligate parasite is a Theileria species the subject may be a cow, buffalo, sheep or goat. Preferably, when the attenuated obligate parasite is T. parva and T. annulata the subject is a cow or buffalo. Preferably, when the attenuated obligate parasite is T. lestoguardi the subject is a sheep or goat.

In a seventh aspect of the invention there is provided an attenuated obligate parasite of vertebrates and arthropods of the second or third aspect, or the pharmaceutical composition of the fourth aspect, for use as a medicament.

In an eighth aspect of the invention there is provided an attenuated obligate parasite of vertebrates and arthropods of the second or third aspect, or the pharmaceutical composition of the fourth aspect, for use in the prevention, amelioration or treatment of an infection of an obligate parasite of vertebrates and arthropods. The infection may be an Apicomplexa infection. The infection may be selected from the group consisting of: malaria, East Coast fever, babesiosis, cryptosporidiosis, cyclosporiasis, cystoisosporiasis and toxoplasmosis.

Preferably, the attenuated obligate parasite is a Plasmodium spp. and the infection is malaria.

Preferably, the attenuated obligate parasite is a Theileria species and the infection is East Coast fever.

In a ninth aspect of the invention there is provided an attenuated obligate parasite of vertebrates and arthropods of the second or third aspect, or the pharmaceutical composition of the fourth aspect, for use as a vaccine.

Preferably, the attenuated obligate parasite is a Plasmodium spp. and the vaccine provides protection against malaria.

Preferably, the attenuated obligate parasite is a Theileria species and the vaccine provides protection against East Coast fever.

In a tenth aspect of the invention there is provided a vaccine comprising an attenuated obligate parasite of vertebrates and arthropods of the second or third aspect, or the pharmaceutical composition of the fourth aspect.

Preferably, the attenuated obligate parasite is a Plasmodium spp. and the vaccine provides protection against malaria.

Preferably, the attenuated obligate parasite is a Theileria species and the vaccine provides protection against East Coast fever.

Preferably, the vaccine comprises a suitable adjuvant.

The attenuated obligate parasite or the pharmaceutical composition of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The attenuated obligate parasite or the pharmaceutical composition of the invention may also be incorporated within a slow- or delayed-release device. Such devices may, for example, be inserted on or under the skin, and the medicament may be released over weeks or even months. The device may be located at least adjacent the treatment site.

In a preferred embodiment, however, medicaments according to the invention may be administered to a subject by injection into the blood stream, muscle, skin or directly into a site requiring treatment. Injections may be intravenous (bolus or infusion) or subcutaneous (bolus or infusion), or intradermal (bolus or infusion).

It will be appreciated that the amount of attenuated obligate parasite or the pharmaceutical composition that is required is determined by its biological activity and bioavailability, which in turn depends on the mode of administration, the physiochemical properties of the attenuated obligate parasite or the pharmaceutical composition and whether it is being used as a monotherapy or in a combined therapy.

The frequency of administration will also be influenced by the half-life of the active agent within the subject being treated. Optimal dosages to be administered may be determined by those skilled in the art, and will vary with the attenuated obligate parasite or the pharmaceutical composition in use, the strength of the pharmaceutical composition, the mode of administration, and the type of parasitic infection. Additional factors depending on the particular subject being treated will result in a need to adjust dosages, including subject age, weight, gender, diet, and time of administration.

Generally, a dose of between 4500 parasites/kg of body weight and 3,375 parasites/kg of body weight, to a maximum of 4 individual administrations, of the attenuated obligate parasite or the pharmaceutical composition of the invention may be used for ameliorating or preventing a parasitic infection, depending upon the active agent used.

Doses may be given as a single administration (e.g. a single injection). Alternatively, the attenuated obligate parasite or the pharmaceutical composition may require more than one administration. As an example, the attenuated obligate parasite or the pharmaceutical composition may be administered as two (or more depending upon attenuated obligate parasite) doses of between 3000 and 5000 parasites/kg (i.e. assuming a body weight of 70 kg). Alternatively, a slow release device may be used to provide optimal doses of the attenuated obligate parasite or the pharmaceutical composition according to the invention to a patient without the need to administer repeated. Routes of administration many incorporate intravenous, intradermal subcutaneous, or intramuscular routes of injection.

Known procedures, such as those conventionally employed by the pharmaceutical industry (e.g. in vivo experimentation, clinical trials, etc.), may be used to form specific formulations of the attenuated obligate parasite according to the invention and precise therapeutic regimes (such as doses of the agents and the frequency of administration).

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of attenuated obligate parasite or the pharmaceutical composition is any amount which, when administered to a subject, is the amount of the aforementioned that is needed to ameliorate or prevent parasite infection.

For example, the attenuated obligate parasite and the pharmaceutical composition of the invention may be used may be between 10000 parasites/kg of body weight and 1000 parasites/kg of body weight, preferably between 5000 parasites/kg of body weight and 3000/kg of body weight, most preferably between 4500 parasites/kg of body weight and 3375 parasites/kg of body weight. The obligate parasite and the pharmaceutical composition of the invention may be applied in a maximum of 4 individual administrations, preferably to a maximum of 2 individual administrations.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. attenuated obligate parasite and of the invention) may be mixed with a vehicle having the necessary compression properties in suitable proportions and compacted in the shape and size desired. The pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The attenuated obligate parasite according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The attenuated obligate parasite of the invention may be prepared as any appropriate sterile injectable medium.

The attenuated obligate parasite and the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The attenuated obligate parasite of the invention and the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

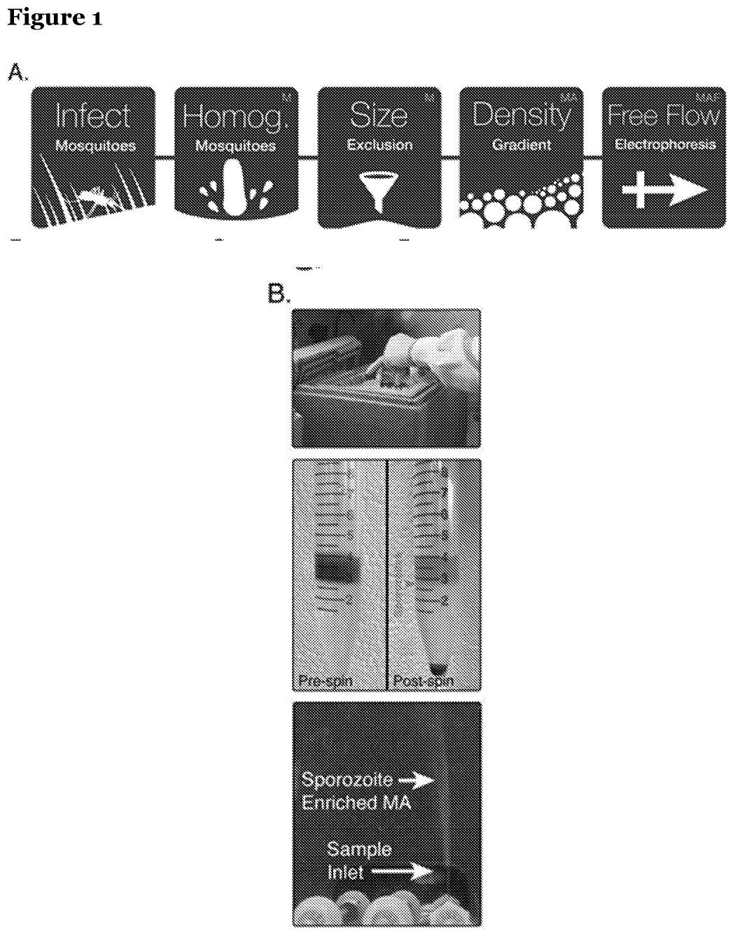

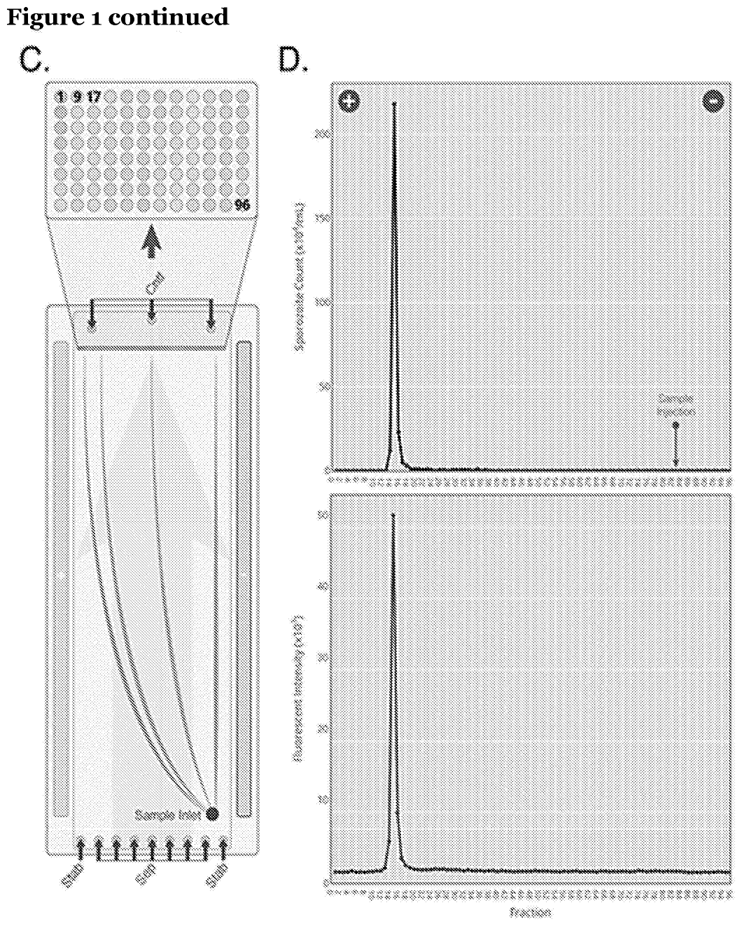

FIG. 1 shows purification of sporozoites from whole mosquitoes using MalPure V1.0. A) Schematic of key steps in the sporozoite purification process. B) Top; mosquito homogenisation step, middle; Accudenz gradient, bottom; FFE separation of MA sporozoites. C) Schematic representation of sample separation by cZE mode. In cZE an electrophoretic buffer is run through a chamber 0.5 mm thick with a voltage applied across the flow. Sample added to the start of the chamber is carried vertically up the length of the chamber (pale blue arrow) as a voltage is applied across, which causes the sample constituents to shift to their isoelectric point (indicated by the four lines), effectively separating across the horizontal length of the chamber. The outflow from the chamber is separated into 96 outlets along the horizontal length of the chamber, separating the sample into 96 fractions which drop into a 96 well plate (rainbow colour used to indicate plate layout of fractions). A counter-flow buffer is applied into the top of the chamber to ensure that the sample leaves via the 96 outlet tubes. A stabilisation buffer with 10-fold molar concentration over the running buffer is run along each side of the chamber to prevent sample running into the electrodes. Separation can be modified by adjusting the buffer, buffer flow rate, chamber voltage and sample injection rate. D) Manual sporozoite count by haemocytometer (top) and fluorescent plate read at 610 emission (bottom) of FFE fractions from a representative MAF sporozoite separation. Point of sample injection indicated by arrow and direction of current indicated by positive and negative symbols.

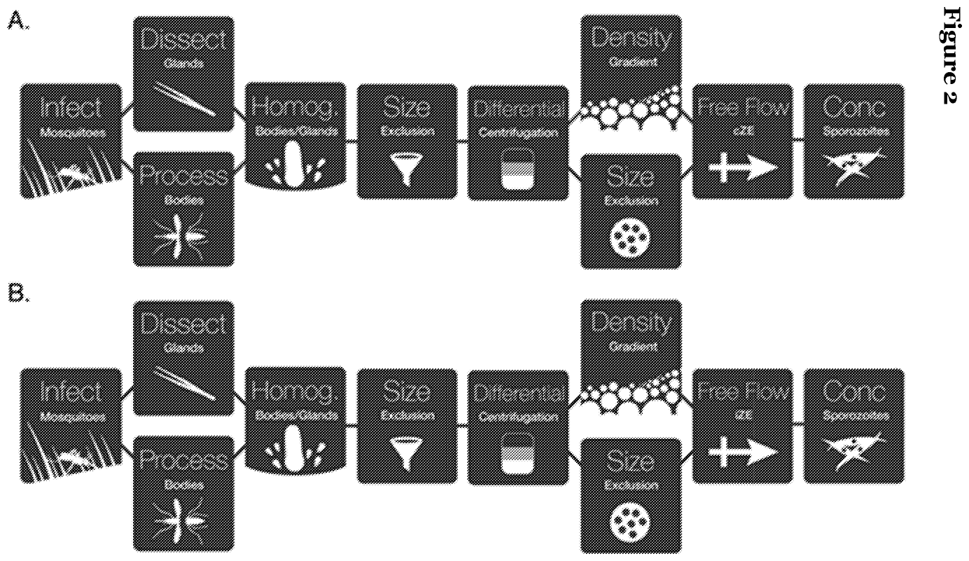

FIG. 2 shows purification of sporozoites form whole mosquitoes using MalPure V1.1 and V2.0. A) Schematic of MalPure V1.0 when modified with some of the advancements from MalPure V2.0. These include the processing of bodies, differential centrifugation and size exclusion. As stated in the next some of these steps are optional depending on your desired output. B) Schematic of MalPure V2.0. As stated in the next some of these steps are optional depending on your desired output. C) Schematic representation of sample separation by iZE mode. In iZE an electrophoretic buffer is run through a chamber 0.2 mm thick. Sample is added to the start of the chamber and carried vertically up the length of the chamber (pale blue arrow). After injection a voltage is then applied across with reduced buffer flow, which causes the sample constituents to shift (indicated by the lines), effectively separating across the horizontal length of the chamber. The outflow from the chamber is separated into 96 outlets along the horizontal length of the chamber, separating the sample into 96 fractions which drop into a 96 well plate (rainbow colour used to indicate plate layout of fractions). A counter-flow buffer is applied into the top of the chamber to ensure that the sample leaves via the 96 outlet tubes. A stabilisation buffer with 10-fold molar concentration over the running buffer is run along each side of the chamber to prevent sample running into the electrodes. Separation can be modified by adjusting the buffer, buffer flow rate, chamber voltage and sample injection rate. D) Manual sporozoite count by haemocytometer of FFE fractions from a representative MAF sporozoite separation. Shows two peaks of sporozoite separation, the pure sporozoite peak is indicated by the arrow (referred to as highly pure fraction throughout).

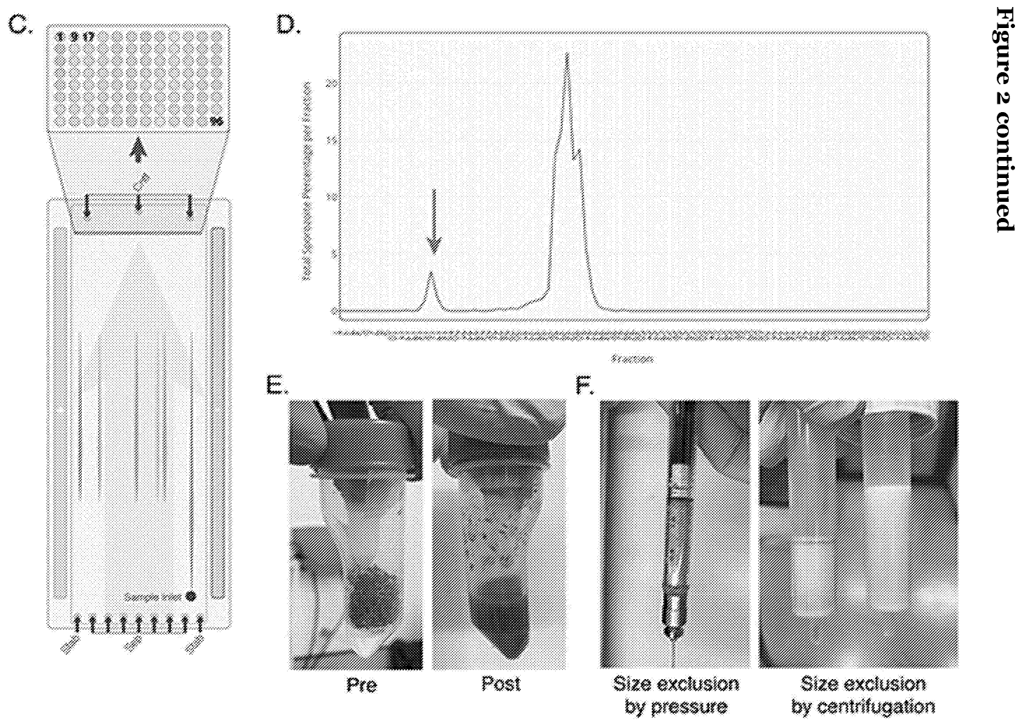

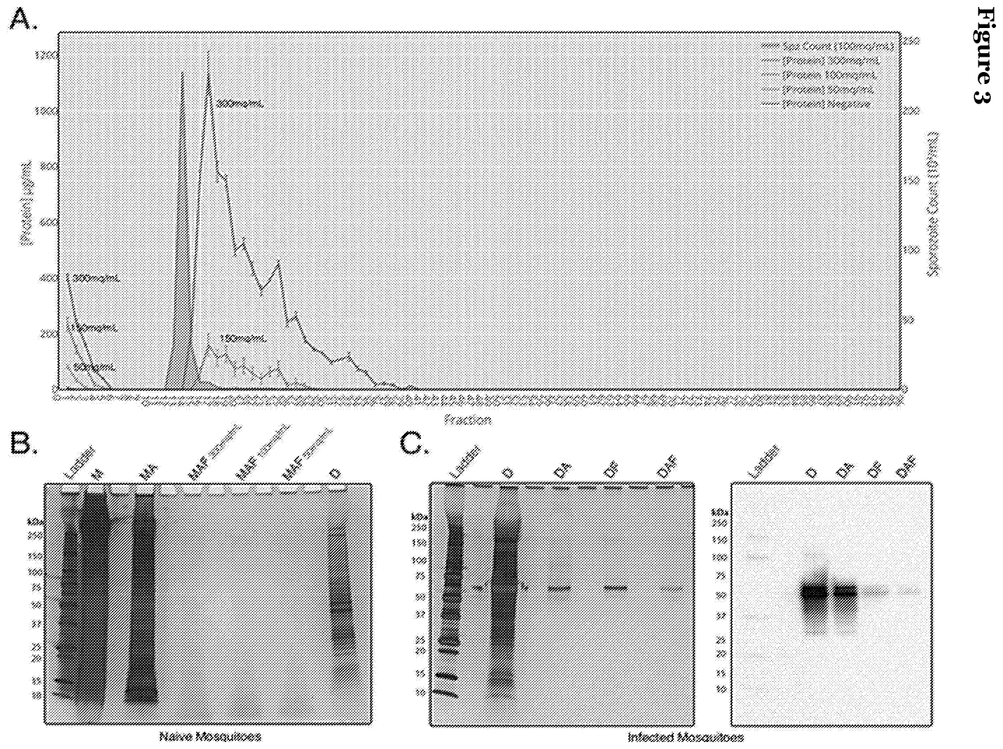

FIG. 3 shows separation of mosquito-associated protein contaminants with MalPure V1.0. A) Protein concentration in each fraction after loading naïve mosquito MA onto the FFE machine at three doses of mosquitoes (MAF). Sporozoite distribution (purple) from infected mosquitos loaded at 100 mq/mL is marked to allow comparison of purification. B) Silverstain of uninfected mosquitoes from each step of purification. Uninfected MAF lanes are from the same fraction as the sporozoite peak fraction identified by running infected mosquitoes at the same time. C) Left; Silverstain of infected mosquitoes from each step of purification when using dissected salivary glands for homogenisation instead of total mosquitoes. Right; western blot against P. berghei CSP from the same samples as the silver stain (left). MAF samples in C) where injected into the FFE machine at 100 mq/mL. All silver stains from reducing SDS-PAGE's with samples normalised by meq with 4 meq's loaded onto each lane. D) Photographs of pelleted sample from M, MA and MAF purification stages. All pellets normalised by meq. E) Brightfield images of sporozoites from MAF and manual SGD with a zoomed in portion. Both treatments normalised to same concentration of spz/mL. F) Brightfield images of each stage of purification from whole mosquito homogenate. All stages diluted to 7×10⁵ sporozoites/mL. G) Identification of tryptic peptide 297-312 from P. berghei CSP protein (Swissprot ID: Po6915) with MASCOT score of 37 by mass spectrometry analysis. All samples normalised to 200 mq/mL.

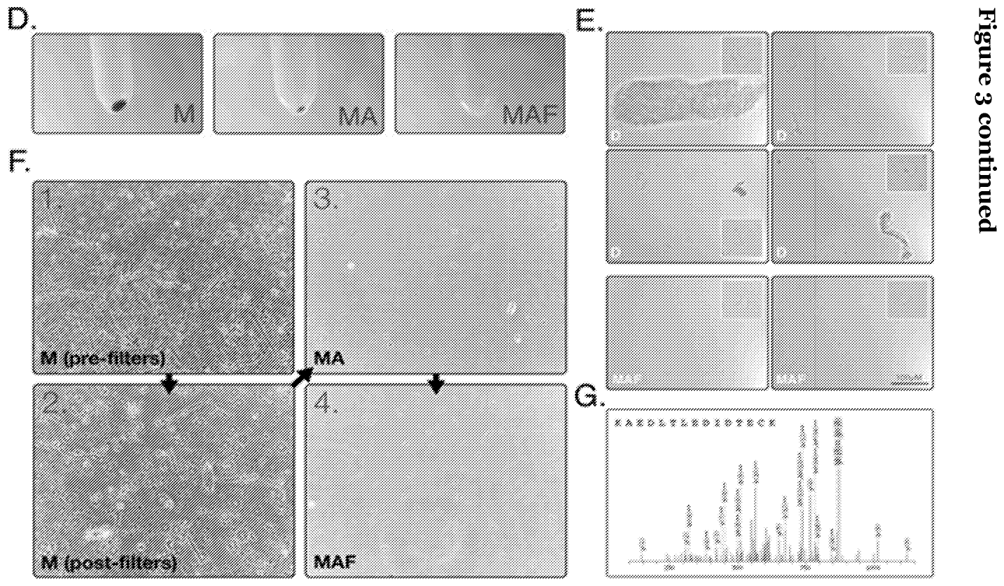

FIG. 4 shows separation of mosquito-associated protein contaminants with MalPure V2.0. A) Protein concentration in each fraction after loading uninfected mosquito MA onto the FFE machine at three doses of mosquitoes (50, 100, 300 mosquitos/mL; mq/mL). Sporozoite distribution from infected mosquitos loaded at that same doses is marked to allow comparison of purification. B) Silverstain of uninfected mosquitoes from each step of purification. Uninfected MAF lanes are from the same fraction as the sporozoite peak fraction identified by running infected mosquitoes at the same time. C) Images of the sporozoite pellet from MA and the highly pure fraction from MAF. D) Brightfield images of sporozoites from MA and the highly pure fraction from MAF.

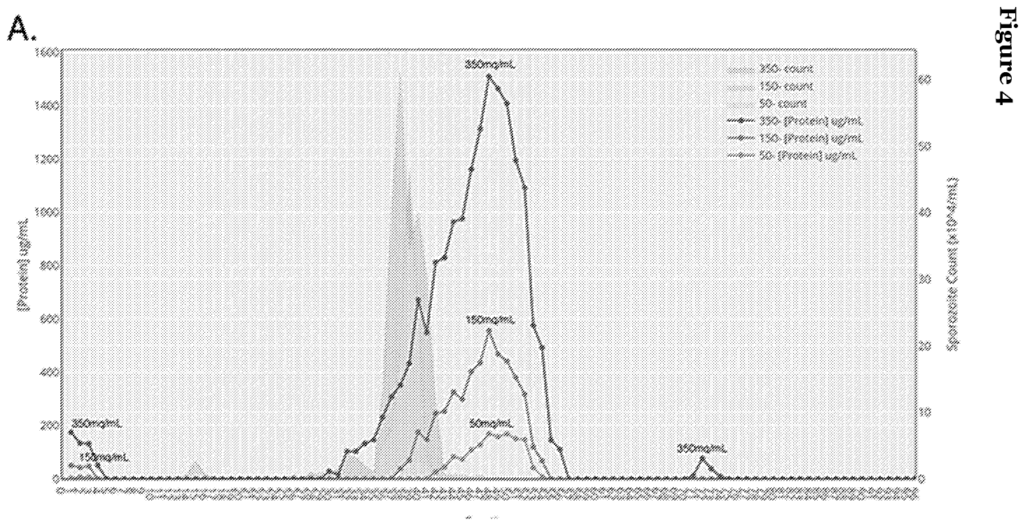

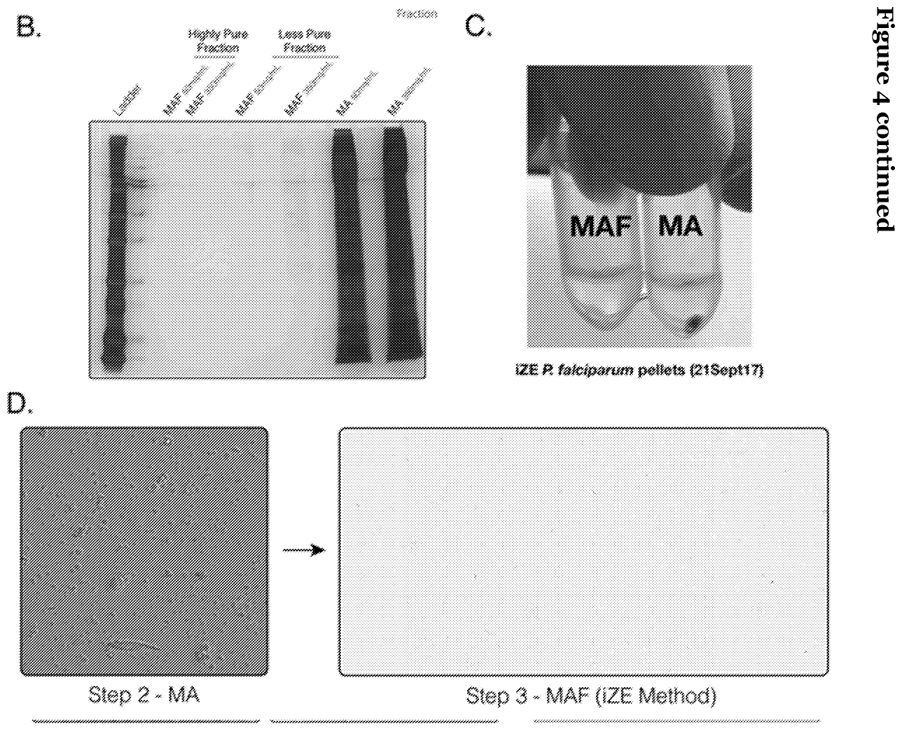

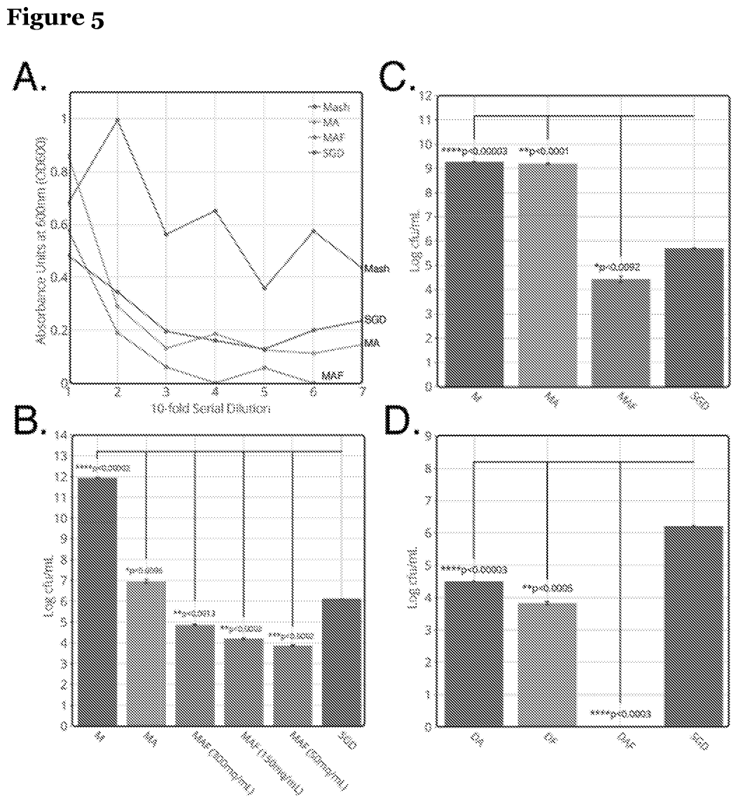

FIG. 5 shows separation of mosquito-associated bacterial contaminants using MalPure V1.0. A) End-point 16 hr serial dilution for each step of MAF purification. Absorbance of samples in TBS was measured at 600 nm (OD600) 16 hr post-inoculation. All growth conducted at 37° C., 300 rpm, using mosquitoes blood fed on uninfected mice 21 days prior to MAF extraction. B) Bacterial growth at different stages from uninfected whole mosquito (M) origin purification. Samples were loaded onto the FFE machine at three different originating mosquito doses. C) Bacterial growth at different stages from infected whole mosquito (M) origin purification. D) Bacterial growth at different stages from infected SGD origin purification. All experiments show the mean of two technical replicates and error bars represent SEM. All treatments compared to dissected by unpaired two tailed t-test using Bonferroni correction (A: *p<0.01, **p<0.002, ***p<0.0002, ****p<0.00002; B-C: *p<0.017, **p<0.0034, ***p<0.00034, ****p<0.000034; D-E: *p<0.025, **p<0.005, ***p<0.0005, ****p<0.000005). Bacterial colony forming units log transformed. Samples normalised by meq (200 mq/mL).

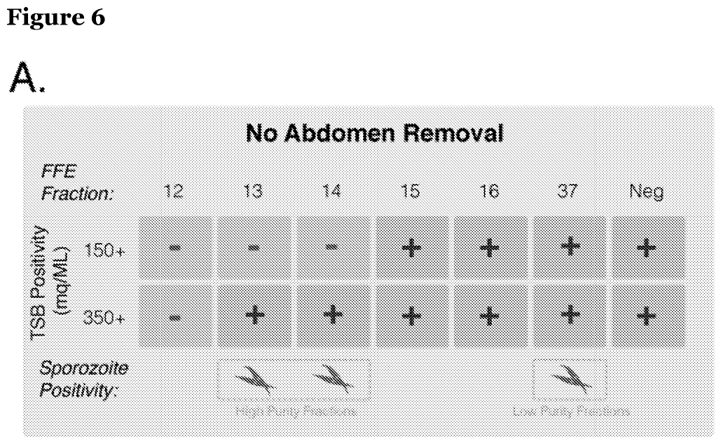

FIG. 6 shows separation of mosquito-associated bacterial contaminants using MalPure V2.0. A) Bacterial positivity of fractions from the high (fractions 13-14) and a representative low (fraction 37) purity fraction, assessed by tryptic soya broth growth. Green indicates bacteria free, red indicates bacterial growth. Infected mosquitoes loaded at 150 and 300 mq/mL onto FFE.

FIG. 7 shows assessment of MAF sporozoites viability with MalPure V1.0. A) Typical movement trails of MAF and SGD sporozoites over 600 frames at 2 Hz. B) Sporozoite gliding motility over 600 frames at 2 Hz with a sliding nine frame average during each motility state over 600 frames at 2 Hz. C) Comparison of the percentage of all sporozoites in each state. Sporozoite tracking represents mean of two independent replicates and six technical replicates with groups compared using an unpaired two-tailed t-test. Bars represent means and error bars the SEM. D) Absolute RT-PCR quantification of parasite HSP70 DNA copies normalised by host HSP60 gene in HepG2 and primary rat hepatocytes. Three independent replicates. E) Manual counts of successful hepatocyte infections in primary rat hepatocytes measured by visual identification of six fields of view over 24 hr time-lapse from three independent replicates. F) Fluorescent image of late stage schizont (52 hr) captured using structured illumination microscope. Blue; nuclei, green; actin, red; mCherry parasite, pink; parasite actin (anti-5H3 (44)). G) Kaplan-Meier survival curve of mice challenged intravenously (i.v.) with increasing doses of sporozoites from MAF. Endpoint classed as 1% parasitaemia. H) Kaplan-Meier survival curve of mice challenged i.v. with 5000 sporozoites from different purification steps. Endpoint classed as 1% parasitaemia, treatments compared by Mantel-Cox statistical test. I) Kaplan-Meier survival curve of mice challenged with 5000 sporozoites obtained by MA purification from different mosquito sources or SGD origin. Death classed as 1% parasitaemia, treatments compared by Mantel-Cox statistical test. J) Sporozoite distribution of infected mosquitoes, average from 85 mosquitoes, two replicates. K) Kaplan-Meier survival curve of mice challenged i.v. with 1000 sporozoites from MaAF purified (MAF from mosquitoes with abdomens removed prior to homogenisation) and SGD origin. Endpoint classed as 1% parasitaemia, treatments compared by Mantel-Cox statistical test.

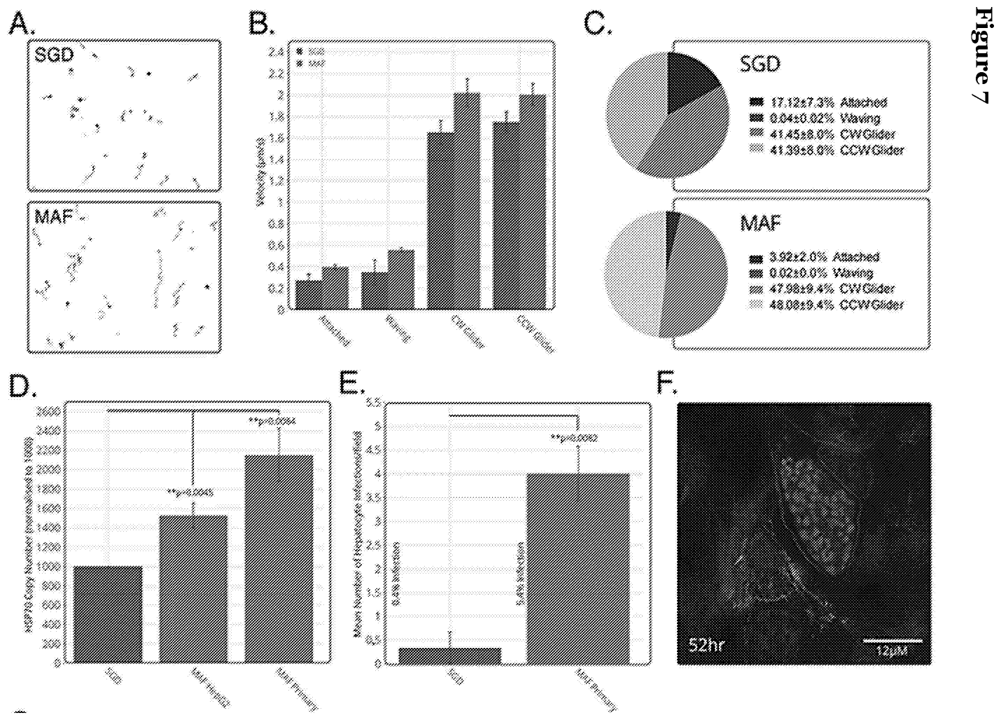

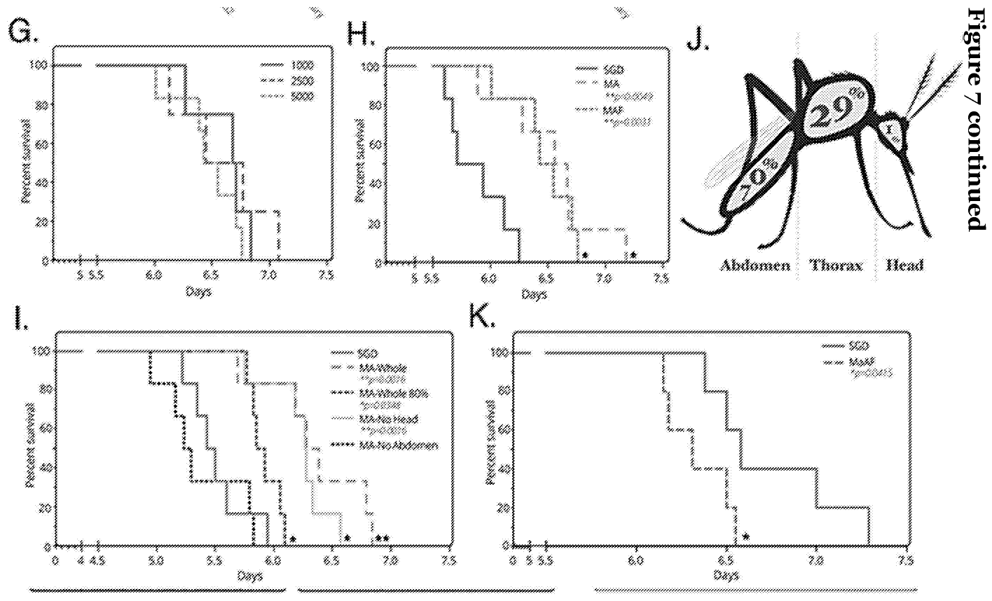

FIG. 8 shows assessment of MAF sporozoites viability with MalPure V2.0. A) Kaplan-Meier survival curve of mice challenged intravenously (i.v.) with three doses of sporozoites from MAF or manual SGD (D). Endpoint classed as 1% parasitaemia. B) Dot plot of data from (A), correlating sporozoite dose with time to 1% parasitaemia. C) Fluorescent images of HCo4 (left) and immortalised primary hepatocytes (right) infected in vitro with P. falciparum sporozoites at 3 hr and 72 hr post infection.

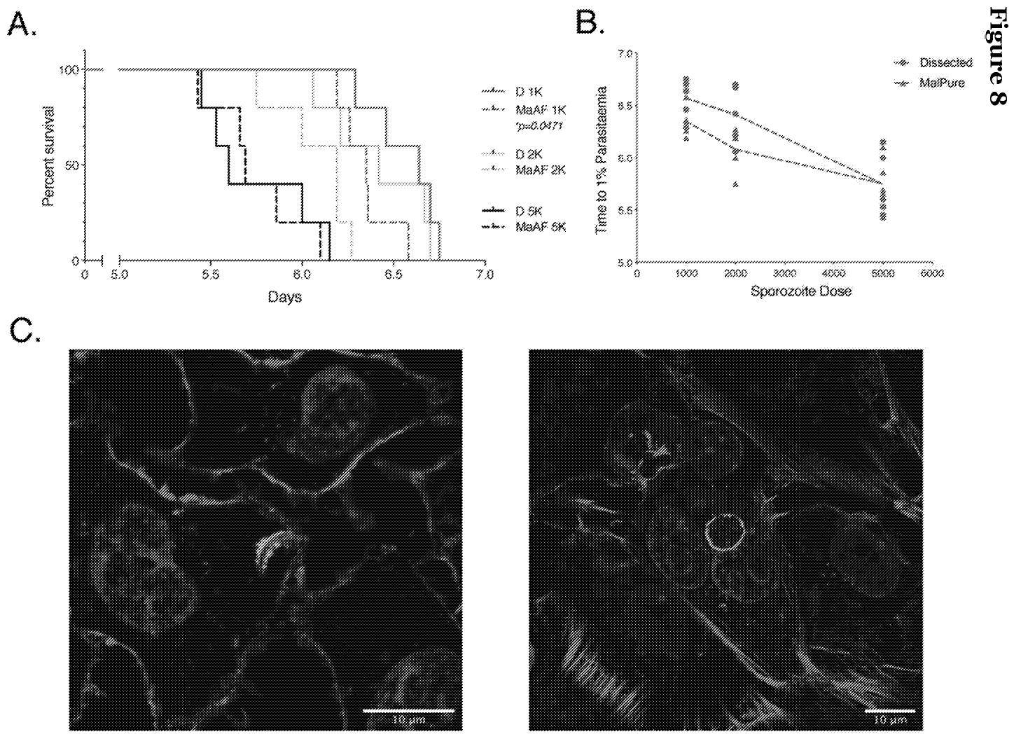

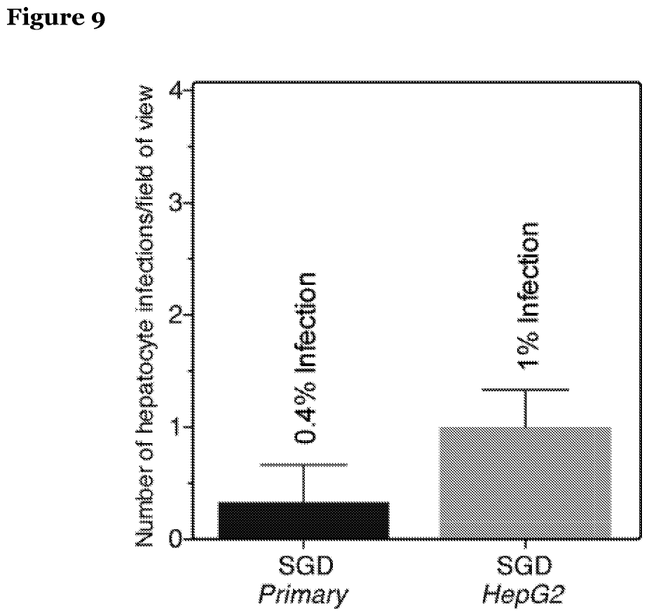

FIG. 9 shows In vitro liver-stage infection rates using salivary gland dissection. Mean percent infection rate of HepG2 and primary rat hepatocytes by manual quantification of six fields of view of three independent replicates, 40 hr post addition of sporozoites from 2 Hz 48 hr time-lapses. Error bars represent SEM. All replicates used mCherry expressing P. berghei sporozoites isolated by hand dissection of salivary glands.

FIG. 10 shows sporozoite distribution in FFE fractions. A) Parasite distribution into FFE fractions when loaded at four different sporozoite doses (spz/mL). Quantification by haemocytometer count. B) Sporozoite distribution based on total percent of sporozoites per fraction for each sporozoite dose.

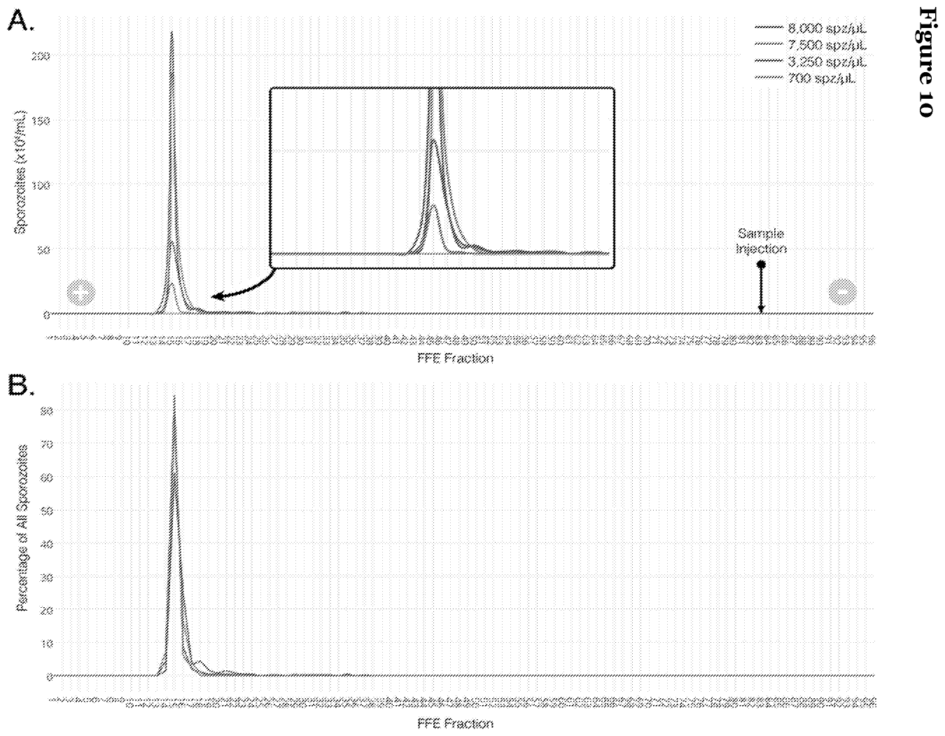

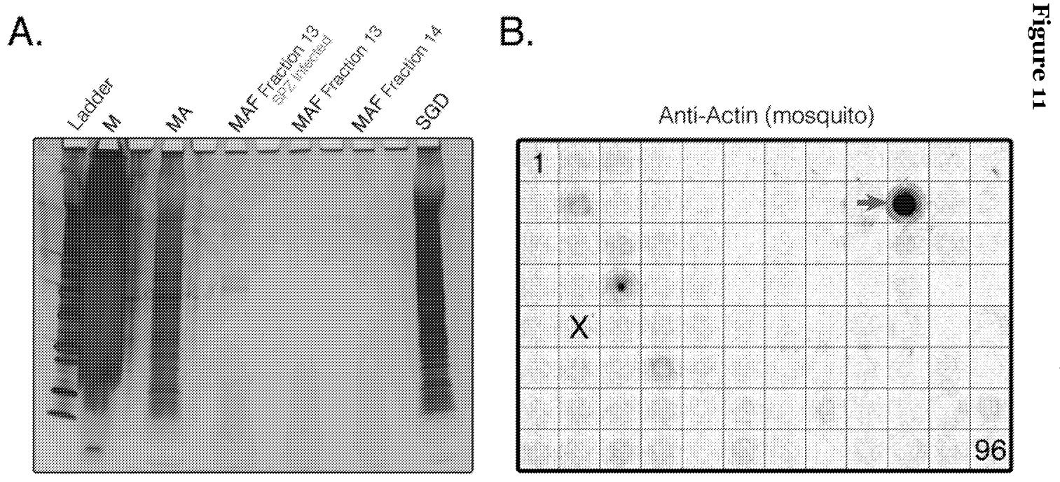

FIG. 11 shows protein purity of sporozoite purification steps. A) Silverstain of uninfected mosquitoes from each step of purification, with the MAF fraction from sporozoite infected mosquitoes (fraction 13) included (labelled SPZ infected on the figure) and MAF fraction 13 and 14 from uninfected mosquitoes. Each lane loaded with 4 meq's. B) Dotblot of all 96 FFE fractions in a plate layout against mosquito actin protein (Sigma Aldrich, A2066). Actin positive fraction indicated by arrow. Sporozoite peak fraction indicated by X Fraction 1 and 96 indicated.

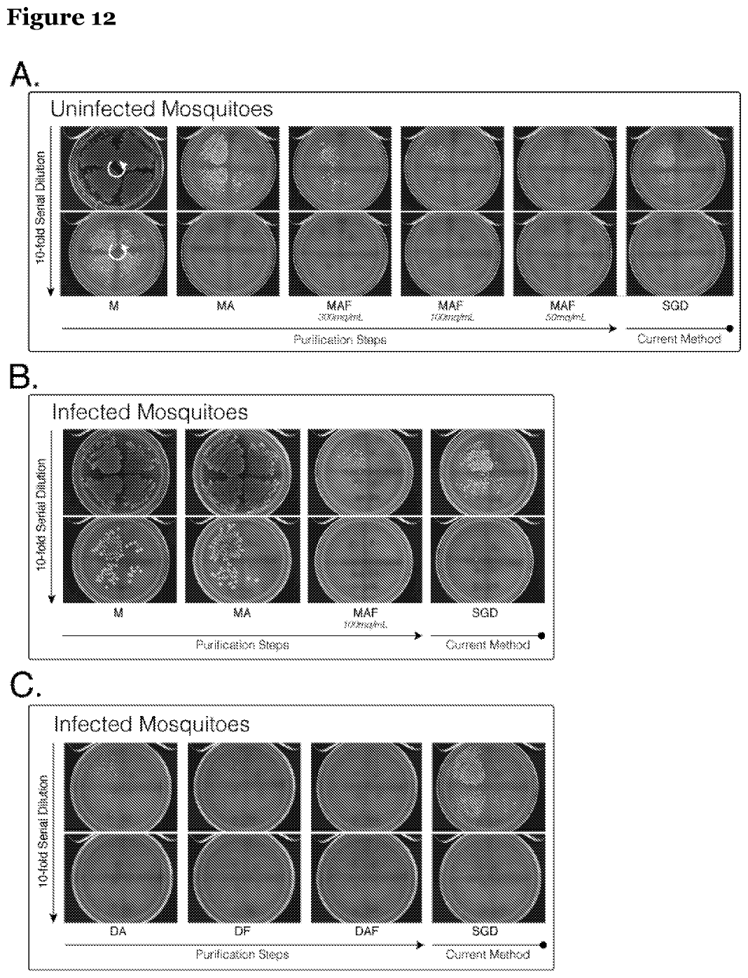

FIG. 12 shows blood plate agar growth of sporozoite purification steps. A) Bacterial growth at different steps of purification from uninfected whole mosquito homogenate. Samples were loaded onto the FFE machine at three different meq's (300, 100, 50 mq/mL). B) Bacterial growth at different purification steps from infected whole mosquito homogenate. C) Bacterial growth at different stages from infected dissected salivary gland homogenate. All samples spread onto blood agar plates in eight, 10-fold serial dilutions running anti-clockwise, with plates separated into 4 quadrants and dilutions starting in the top right quadrant (indicated by white arrows).

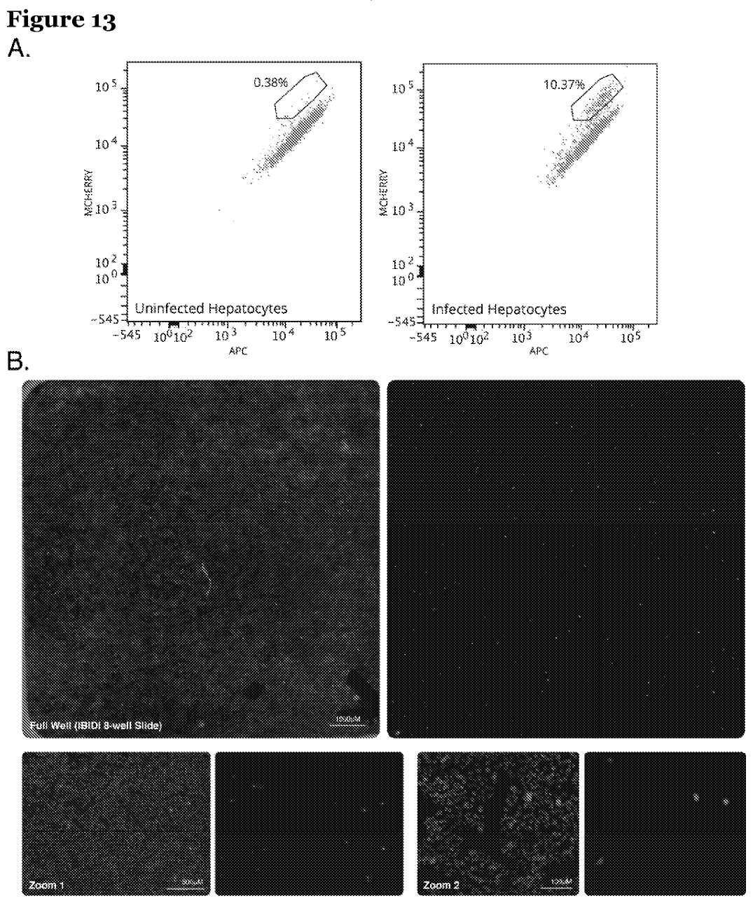

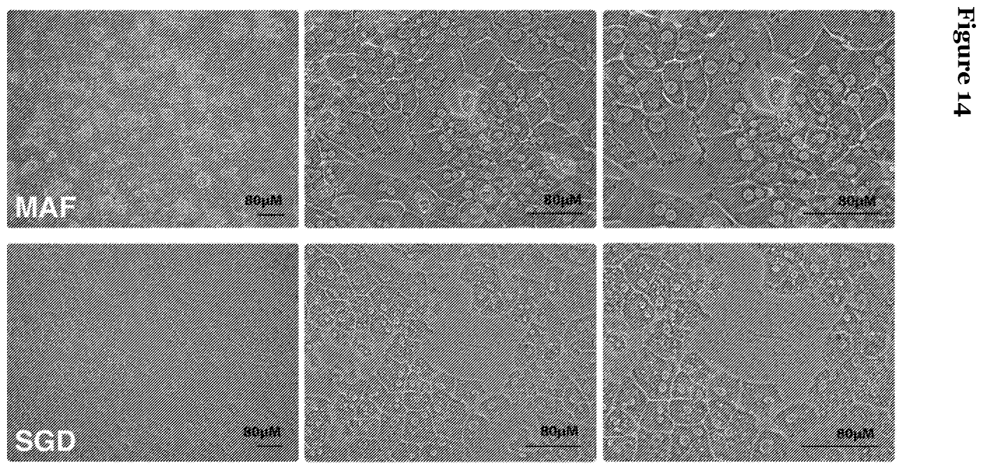

FIG. 13 shows P. berghei 52 hr liver schizonts. A) Flow cytometry quantification of mCherry expressing transgenic P. berghei infected primary rat hepatocytes 36 hr post addition of sporozoites (total 2808 cells in treatment, 3906 cells in control). Data representative of three technical replicates. B) Upper: full well view of HepG2 cells fixed 52 hr post infection and stained with anti-5H3⁷⁰ parasite actin antibody. Lower: zoomed in view of well. Blue; nuclei, pink; parasite actin (anti-5H3⁷⁰).

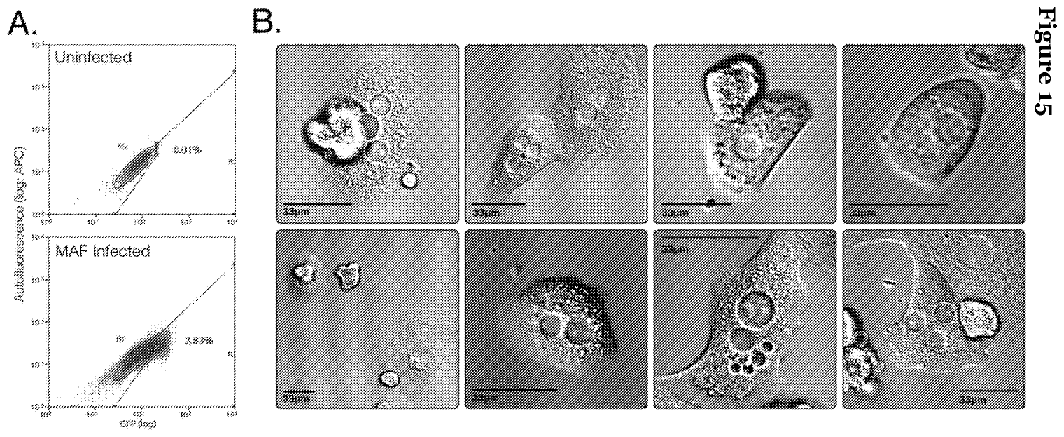

FIG. 14 shows sporozoite-associated morphological changes in primary rat hepatocytes. Brightfield images of primary rat hepatocytes 20 hr after addition of sporozoites obtained by either MAF (top row) or SGD (bottom row). Cultured in 1% P/S.

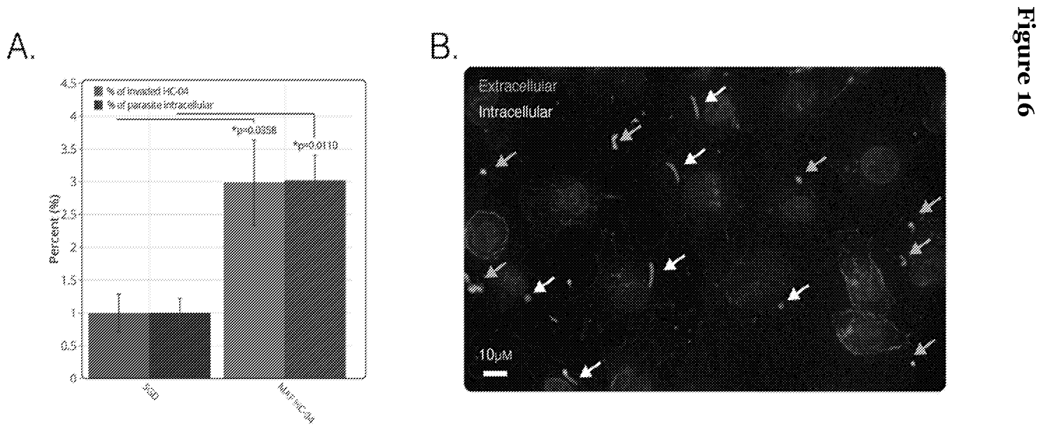

FIG. 15 shows Ex-vivo development of MAF purified P. berghei sporozoites in rat primary hepatocytes. A) The ability of MAF sporozoites to infect hepatocytes in vivo but develop ex vivo was investigated. Hepatocytes were extracted by perfusion of livers that were collected from rats 14 hr after intravenous injection of sporozoites into rats. These rats were infected with a total of 3×10⁷ GFP-expressing P. berghei sporozoites purified by MAF (whole mosquitoes) from 400 mosquitoes. Infected hepatocytes from these rats were collected by flow-sorting and subsequently plated and incubated for a period of up to 30 hr. Flow sorting identified 2.83% GFP-positive cells in the extracted, perfused liver cell population. B) Fluorescent images of GFP positive cells collect by flow sorting 24 hours after plating.

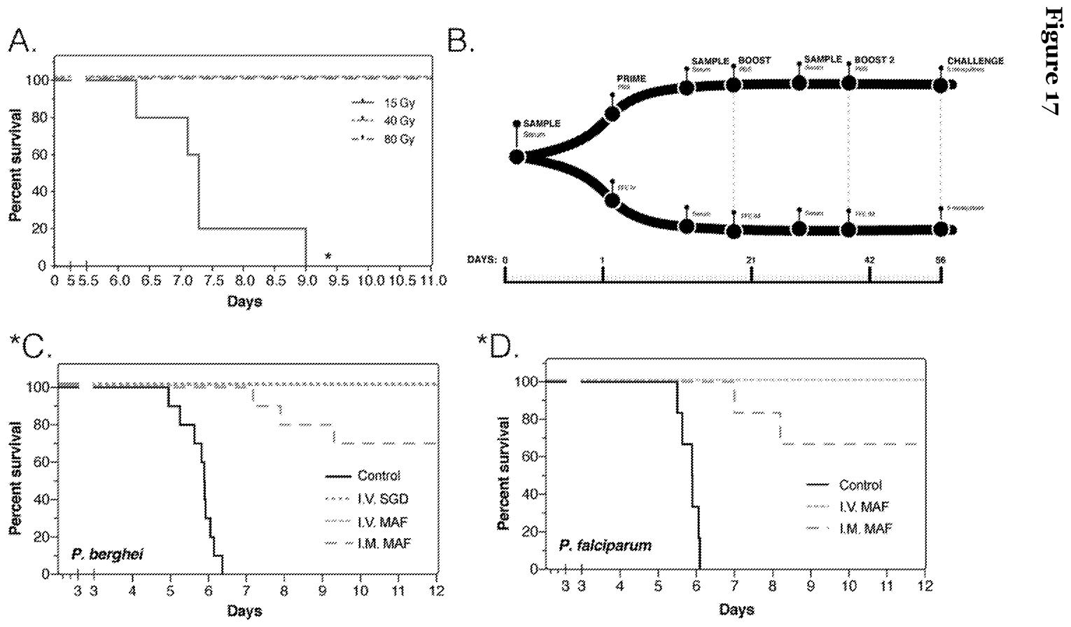

FIG. 16 shows P. falciparum infectivity in vitro. A) Counts of successful invasions of P. falciparum sporozoites four hours post infection in HC-04 at a ratio of 1:5 cells to sporozoites. SGD treatment normalised to 1. Sporozoites stained for CSP to determine intracellular or extracellular location. B) Immunoflourescent staining of HC-04 cells with fixed four hours after infection with P. falciparum sporozoites and stained with anti-CSP (extracellular=green+red, intracellular=red only), DAPI for nuclear material (blue) and phalloidin for actin (purple).

FIG. 17 shows that purified sporozoites are a viable vaccine. A) Kaplan-Meier survival curve of mice challenged i.v. with 1000 P. berghei sporozoites from MaAF purified and gamma irradiated. Endpoint classed as 1% parasitaemia. B) Schematic of vaccination regime used. Sporozoites were either from SGD or MaAF origin, then gamma irradiated. C) Immunisation i.v. or i.m. of mice with irradiated P. berghei sporozoites from either manual salivary gland (SGD) dissection or MaAF. Mice given three immunisations of 40 k sporozoites, two weeks apart followed by challenge with five infectious mosquito bites. D) Immunisation i.v. or i.v. of mice with irradiated P. falciparum sporozoites from MaAF). Mice given three immunisations of 40 k sporozoites, two weeks apart followed by challenge with five infectious mosquito bites.

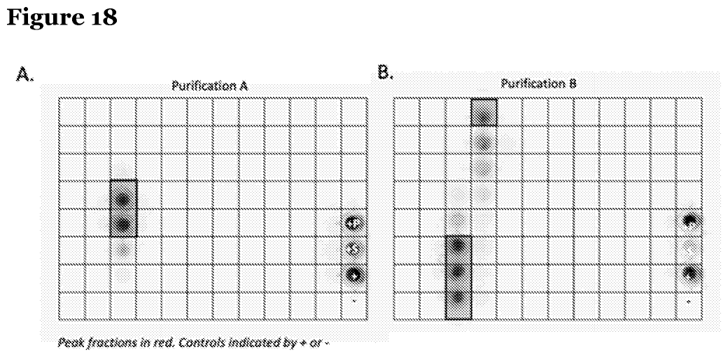

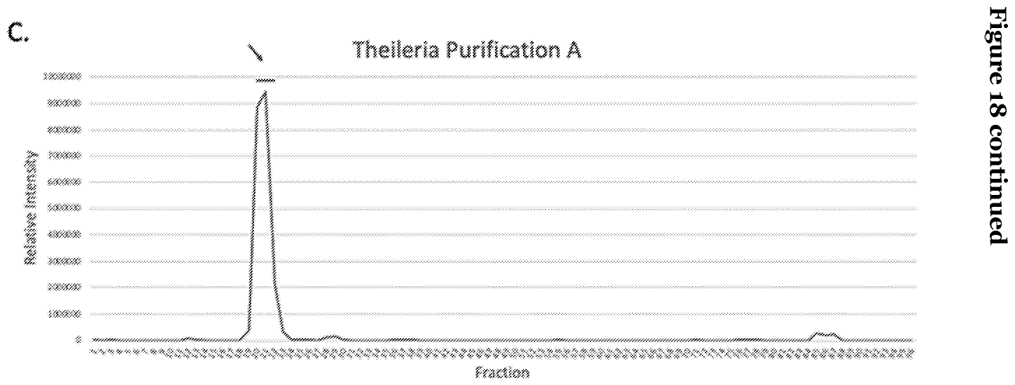

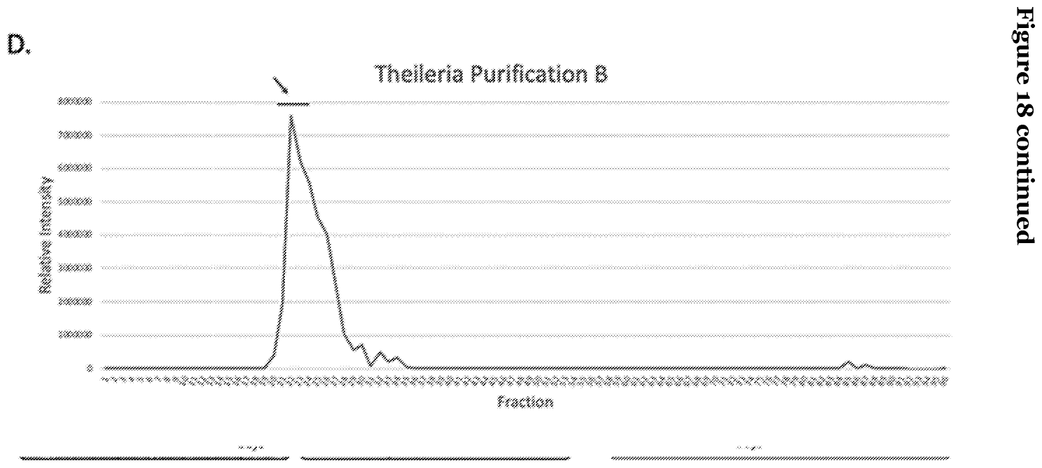

FIG. 18 shows dot blot of fractions from isolation methods A (a) and B (b) probed respectively with antibodies against T. parva schizonts (A) and T. parva hsp70 (B). Densitometry plots of each separation method respectively (c, d).

### EXAMPLES

Materials and Methods

Mosquito Maintenance:

An. stephensi mosquitos used for experiments were raised at 28° C., 70% relative humidity with a 12 hr light cycle. Larvae were fed with fish pellets and adults maintained on 10% fructose.

P. berghei Maintenance and Infection:

Two transgenic P. berghei ANKA lines were used in this study that express either mCherry or GFP under control of the uis4 promoter. For infection of mice, cryopreserved parasitized RBC's (day five) were thawed and injected into naïve Balb/c mice by the intraperitoneal (i.p.) route and An. stephensi mosquitos allowed to feed on anesthetised mice with 1-2% blood-stage parasitaemia. 7-10 days later these mosquitoes were allowed to take an additional bloodmeal on naïve Balb/c mice to increase sporozoite yields. Blood-fed mosquitos were maintained at 19° C. at 70% relative humidity for 19-22 days before sporozoites were extracted.

Manual Salivary Gland Dissection (SGD):

Mosquitoes were sedated on ice for 10 min, then placed on a glass slide with 100 μL complete Schneider's Drosophila medium (1% FBS, 4° C., NaHCO₃ free, Pan-Biotech) and whole salivary glands removed by gentle separation of the head using micro-forceps. Both sets of glands were gently cleaned to remove other tissues then placed into a glass dounce tissue grinder on ice using 2 μL fresh medium. The glass slide was cleaned between each dissection. Each dissection took approximately (45-90 sec) and was carried out for no more than 2-3 hr maximum to reduce loss of infectivity. To release sporozoites the salivary glands were homogenised with three gentle but firm grinds using the pestle. The sample was transferred to protein lo-bind Eppendorf (used to prevent loss of sporozoites by adhesion to plastic-ware) and mixed well before a sample was added to a haemocytometer and the average of four 16 square fields counted. Sample was diluted if too concentrated to accurately count.

Homogenisation

Mosquitoes sedated on ice were placed in a Petri dish with 2 mL (per 400 mosquitoes) complete Schneider's Drosophila media and gently homogenised with the end of a 10 mL syringe barrel for 30-60 sec. Liquid was removed and collected in a 50 mL tube. A further 1.5 mL media was added to the petri dish, gently homogenised and repeated once more. Alternatively, sedated mosquitoes were added to C/M-tube (Miltenyi Biotec) with complete Schneider's Drosophila media (approximately 6-8 mL/300 mosquitoes) and homogenised using gentleMACS (Miltenyi Biotec). Optionally, prior to homogenisation regions of the mosquito were removed depending on the maturilty level of sporozoite required. For example, if mature sporozoites are required the abdomen is removed prior.

Size Exclusion and Differential Centrifugation:

The homogenate is subsequently passed through a 100 μM filter and the filter washed with 1 mL media. This was repeated for the 70, 40 and 20 μM filters. In later purification revisions a 10 μM filter was also included (M). All steps were carried out on ice. One some occasions the product was centrifuged at 15×g for 1-5 min at 4° C. and the pellet discarded.

Density Gradient and Size Exclusion:

1 mL homogenate was loaded onto a 3 mL Accudenz cushion (4° C.) in a 15 mL centrifuge tube and centrifuged (2500 xg, 4° C.) as per reference (22). Subsequently, 400 μL was taken from the sporozoite enriched boundary (at the 3 mL mark). 1 mL aliquots of Accudenz sporozoites were put into 2 mL protein lo-bind eppendorfs (Eppendorf), made up to 2 mL with complete Schneider's media and centrifuged (12,000 xg, 4° C., 3 min). The resultant pellet was re-suspended in complete Schneider's media (MA).

Alternatively, sample was passed through a sephadex G15-medium column. Firstly, sephadex was hydrated in 3% sodium citrate 50:50 v/v and left at 4° C. overnight. He next day either a glass syringe of a PD-10 column was packed with hydrated sephadex to approximately 3-6 cm height. Homogenate was applied to the column and either pushed through the syringe or centrifuge in the PD-10 column (800-5000 rpm, 1-5 min). The eluted product was centrifuged as above and resuspended in complete Schneider's media (MA).

If the sample was then to be used for free-flow electrophoresis (FFE) it was re-suspended to a mosquito equivalent (ME) of 200 mq/mL (mosquitoes/mL; unless stated otherwise), which was based on the original number of whole mosquitoes homogenised and the final volume this was in. The resultant sporozoite suspension was in most cases was further purified by FFE.

Free-Flow Electrophoresis—Continuous Zone Electrophoresis:

Prior to sporozoite extraction the FFE machine (FFE Service GmbH) was setup for continuous ZE (cZE) using a 0.5 mm ZE spacer, 0.8 mm filter paper. A separation buffer of 10 mM triethanolamine (TEA), 10 mM glacial acetic acid (HAc) and 250 mM sucrose was used with a stabilisation buffer of 100 mM TEA, 100 mM HAc and 250 mM sucrose injected into the separation chamber at 300 mL/hr for BD instruments or 150 mL/hr for FFE Service instruments. Electrodes were kept in 100 mM TEA, 100 mM HAc and 250 mM sucrose with a voltage of 750V and current and power limit of no greater than 250 mA and 200 W respectively for BD instruments or 900 v, max 250 mA and max 200 W for FFE Service instruments. Flow rate and voltage could be varied +/−50 mL/hr and 100V respectively. MA sample was mixed 1:1 with separation buffer (now at 100 mq/mL) and injected into the separation chamber at the cathode end at a rate of 1600 μL/hr and fractions collected 14 min after injection started and stopped 14 min after sample finished. Fractions were collected in 2 mL protein lo-bind deepwell plates (Eppendorf) containing 400 uL complete Schneider's medium. The peak sporozoite fraction(s) was identified by a haemocytometer and centrifuged in 2 mL protein lo-bind tubes (max, 4° C., 3 min) and the pellet re-suspended in 100-500 μL complete Schneider's media (MAF). To compare purification stages all samples were re-suspended to the same ME. FFE ME dose was calculated based on the volume collected in the peak fraction.

Free-Flow Electrophoresis—Interval Zone Electrophoresis:

Prior to sporozoite extraction the FFE machine (FFE Service GmbH) was setup for interval ZE (cZE) using a 0.2 mm ZE spacer, 0.4 mm filter paper. A separation buffer of 10 mM triethanolamine (TEA), 10 mM glacial acetic acid (HAc) and 250 mM sucrose was used with a stabilisation buffer of 100 mM TEA, 100 mM HAc and 250 mM sucrose injected into the separation chamber at 120 mL/hr. Electrodes were kept in 100 mM TEA, 100 mM HAc and 250 mM sucrose. Chamber was precoated with HPMC. MA sample was mixed 1:4 with separation buffer (now at 150 mq/mL) and injected into the separation chamber at the cathode end at a rate of 1000-1800 μL/hr. After 50 seconds the flow rate was changed to 20 mL/hr and voltage applied at 60 seconds (1200V, 120 W, 150 mA). At 4 min, 30 sec the voltage was stopped and flow rate returned to 120 mL/hr before fractions were collected at 6 min until 7 min 55 sec. Fractions were collected in 2 mL protein lo-bind deepwell plates (Eppendorf) containing 400 uL complete Schneider's medium. The peak sporozoite fraction(s) was identified by a haemocytometer and centrifuged in 2 mL protein lo-bind tubes (max, 4° C., 3 min) and the pellet re-suspended in 100-500 μL complete Schneider's media (MAF). To compare purification stages all samples were re-suspended to the same ME. FFE ME dose was calculated based on the volume collected in the highly pure peak fraction. Alternatively, a BISTRIS buffer system was used with varying settings (see Table 1 below).

TABLE 1
Settings of Interval Zone Electrophoresis.
Interval Zone Electrophoresis - TEA/Ac

| System | FFE Service System (2013) | |
|---|---|---|
| Protocol | iZE, 0.2 mm | 1200 V, <250 mA, <200 W, 10° C. |
| | Spacer, | HPMC Chamber Coating. |
| | Horizontal | 220/20 mL/hr |
| Buffers | Separation/ | 10 mM TEA, 10 mM Ac, 250 mM |
| | Counterflow | Sucrose, pH 7.4 |
| | Stabilisation | 100 mM TEA, 100 mM Ac, 250 mM |
| | | Sucrose, pH 7.4 |

| | |
|---|---|
| Injection | 150 mosquitoes/mL (mq/mL), 1:4 Schneiders:seperation |
| | buffer |
| Timings | Start Injection: 0 sec |
| | Stop Injection: 50 sec |
| | Voltage On: 60 sec |
| | Voltage Off: 4 min, 30 sec |
| | Start Collection: 6 min |
| | Stop Collection: 7 min, 55 sec |

Interval Zone Electrophoresis - BISTRIS

| System | FFE Service System (2013) | |
|---|---|---|
| Protocol | iZE, 0.2 mm | 420 V, <250 mA, <200 W, 10° C. |
| | Spacer, | HPMC Chamber Coating. |
| | Horizontal | 220/20 mL/hr |
| Buffers | Separation/ | 30 mM NaCl, 40m M BISTRIS, |
| | Counterflow | 20 mM EPPS, 170 mM Sucrose, |
| | | 10 mM Glucose, pH 7.4 |
| | Stabilisation | 150 mM Na₂SO₄, 40 mM BISTRIS |
| | (Anode) | 20 mM EPPS, pH 7.4 |
| | Stabilisation | 300 mM NaCl, 40 mM BISTRIS, |
| | (Cathode) | 20 mM EPPS, 75 mM Sucrose, pH |
| | | 7.4 |
| | Electrode | 200 mM Na-acetate |
| | (Anode) | |
| | Electrode | 100 mM NaCl, 100 mM HCl, |
| | (Cathode) | 200 mM Imidazol |

| | |
|---|---|
| Injection | 150 mq/mL injection, 1:4 Schneiders:separation buffer |
| Timings | Start Injection: 0 sec |
| | Stop Injection: 47 sec |
| | Voltage On: 60 sec |
| | Voltage Off: 6 min, 37 sec |
| | Start Collection: 8 min, 25 sec |
| | Stop Collection: 10 min, 36 sec |

Hepatocyte Culture:

Tissue culture plates were pre-coated overnight or using plasma-treatment with a 0.1M bicarbonate buffer (pH9.4) (50) of collagen I, collagen IV, fibronectin and laminin (Sigma-Aldrich; 1 μg/cm²). Human HepG2 hepatoma cell lines were maintained in complete DMEM (10% FBS, 100 penicillin/streptomycin, 5% L-glutamine; Sigma-Aldrich) at 37° C. with 5% CO₂. HCO4 hepatoma cell lines were maintained in DMEM/F12 medium (10% FBS, 1% penicillin/streptomycin, 5% L-glutamine, 15 mM HEPES; 1.15% Bicarbonate; Sigma-Aldrich). A confluent mono-layer was maintained using a 18G syringe needle. Corning Hepatocells were plated and maintained in Corning Hepatocyte Medium (10% FBS, 100 P/S; Corning). To obtain primary hepatocytes male Wistar rats [Crl:CD(SD), strain ooi] were anesthetised and a 21G cannula was inserted into the hepatic portal vein and secured using tissue adhesive (3M). Liver perfusion medium (Thermo Sci; 37° C.) was pumped through the cannula at 10 mL/min using a peristaltic pump and once the liver started to lighten (within 30 sec) the speed was adjusted to 20 mL/min. Subsequently the inferior vena cava was cut and over the next 5 min blocked 2-3 times and the pump increased to 40 mL/min. Following successful perfusion, the media was exchanged for liver digest medium (Thermo Sci; 37° C.) and the same blocking procedure carried out for 8 min. The liver was subsequently transferred quickly to 4° C. complete DMEM on ice, the liver disrupted and the passed through 100 μM cell strainers. The cell suspension was washed twice (50 xg, 5 min, 4° C.) with a final re-suspension into 19 mL complete DMEM and 20 mL sterile isotonic percoll (SIP; 90% percoll, 10% 10×PBS) and centrifuged (1.06 g/mL, 100 xg, 10 min, 40 C) to remove debris and dead cells (percoll purification modified from reference (51)). The pellet was washed in complete DMEM and used to seed plates. Importantly the plates were not moved for 30 min to allow the cells to adhere evenly across the plate. They were then transferred to an incubator (37° C., 5% CO₂) for 1-2 hr before medium was exchanged with serum-free hepatocyte growth medium (Promocell) which was exchanged every 12-15 hr.

Sporozoite Motility Assessment

Sporozoites were added to 37° C. complete DMEM and centrifuged (1,500 rpm, 4 min) in glass bottom tissue culture plates to sediment sporozoites. Fluorescent images were captured at 2 Hz for 600 frames at 20× magnification. Motility was assessed using the ToAST ImageJ plugin (52).

Murine Sporozoite Challenge

P. berghei sporozoites were extracted from infected mosquitoes using one of the described methods and diluted in complete Schneider's Drosophila medium (1% FBS, 4° C.). Mice were placed in a 37° C. heat-box for 10 min prior to injection of 50 μL intravenously (i.v.) into either lateral tail vein of restrained mice. From day five parasitaemia was monitored by thin-blood film until three days of positive smears were obtained, mice were then sacrificed. Time to 1% parasitaemia was then calculated by linear regression. If parasites were not detected by day 14 the mice were sacrificed.

In Vitro Hepatocyte Challenge

In vitro sporozoite challenges were carried out on hepatocytes 24 hr after plating. Sporozoites in 4° C. complete Schneider's Drosophila media were diluted in pre-warmed (37° C.) complete DMEM (for HepG2; Sigma-Aldrich) or primary hepatocyte medium (for primary hepatocytes; Promocell) to achieve a desired ratio of sporozoite to hepatocyte (usually 1:1 or 1:2) and the culture media was exchanged with the sporozoite media. Cell cultures were carefully returned to the incubator to prevent swirling and an uneven distribution of sporozoites. Media was then no-longer exchanged for the remainder of the experiment.

Ex Vivo Hepatocyte Challenge

Rats were i.v. challenged with 30 million GFP transgenic sporozoites and 14 hr later hepatocytes extracted by liver perfusion (above). Infected (GFP positive) hepatocytes were sorted (MoFlo) and plated for up to 30 hr.

Bacterial Contaminant Quantification

To assess the sterility of each purification step, tryptic soya broth (TSB; Oxoid) was inoculated with samples normalised by meq and absorbance at 600 nm measured after 16 hr incubation at 37° C. Alternatively, samples normalised by meq were serially diluted in PBS and spread on blood-agar plates incubated overnight at 37° C. Negative growth was confirmed by a further 24 hr incubation.

Protein Purity Quantification

For western blotting, sample was lysed using RIPA buffer with protease inhibitor cocktail (Sigma-Aldrich), protein concentration normalised using Pierce BCA protein assay kit (Thermo Scientific) and sample loaded onto a 12% TGX SDS-PAGE gel using reducing Laemmli buffer and transferred by semi-dry transfer onto a PVDF membrane (Bio-rad Laboratories). P. berghei CSP protein was probed using the 3D11 monoclonal (53) and detected using HRP chemiluminescence. Total protein concentration of purified mosquito sample was assessed in SDS-PAGE gels using Pierce silver stain kit (Thermo Scientific) or in solution using a Pierce BCA protein assay kit (Thermo Scientific). Dot blots were conducted on FFE fractions by loading 200 uL of each fraction onto a multiscreen-IP plate (0.45 μM, Millipore) pre-activated with methanol and incubated overnight (4° C.) before probing and detection of anti-mosquito actin (Sigma, A2066) similar to western blotting using HRP and ECL. Alternatively, protein contaminants were assessed using liquid chromatography tandem-mass spectrometry (LC-MS/MS) with prior sample preparation in 6M urea, 100 mM tris (pH7.8), 5 mM dithiothreitol, 20 mM iodoacetamide with subsequent trypsin digestion overnight and desalting. Mass spectrometry output data was analysed using the Mascot algorithm (V2.4) and UniProt database.

Flow Cytometry

Flow cytometry was carried out using an LSRII (Becton Dickson). Hepatocytes were washed three times in ix PBS and removed by gentle cell scraping. Hepatocytes were gated for single cell using FSC-H versus FSC-A and mCherry-P. berghei infected cells detected in the PE-Texas Red channel by comparing to APC channel auto fluorescence. Uninfected hepatocytes were run as controls. GFP-expressing P. berghei infected primary hepatocytes were sorted using a MoFlo cytometer (Beckman) gated for GFP positive single cells.

Fluorescent Microscopy

Timelapse imaging was carried out using 1.5 mm glass bottom dishes/plates (Mattek) on a fluorescent microscope with LED fluorescence light source at 2 Hz (Ludwig Institute, Oxford). Late stage schizonts captured using structured illumination microscopy with a Zeiss, Elyra (Imperial College London, FILM facility).

Quantitative PCR

DNA was extracted from cultures using phenol-chloroform-isopropanol precipitation and re-suspended in molecular grade water. Nucleic acid concentration was determined using a Qubit fluorometer (Thermo Scientific). Quantification of P. berghei hepatocyte infection density based in absolute genome copies was determined using a standard curve plasmid containing a 271 bp fragment from murine heat shock protein (HSP) 60 (Ensembl: ENSMUST00000027123) housekeeping gene and a 176 bp fragment from P. berghei HSP70 gene (PBANKA_071190). 100 ng of DNA template was amplified using SsoAdvanced Universal SYBR green supermix (Bio-Rad Laboratories) run on a CFX Connect RT-PCR machine (Bio-Rad Laboratories) as per manufacturers standard protocol and parasite genome numbers determined using linear fit normalised to HSP60 housekeeping (HSP60 HepG2 F.: GACCAAAGACGATGCCATGC—SEQ ID No:1, R: GCACAGCCACTCCATCTGAA—SEQ ID No: 2; HSP60 Rat F: TGGAGAGGTCATCGTCACCA SEQ ID No: 3, R: CACAGCTACTCCATCTGAGAGT—SEQ ID No:4; HSP70 P. berghei F: AGGAATGCCAGGAGGAATGC—SEQ ID No: 5, R: AGTTGGTCCACTTCCAGCTG—SEQ ID No: 6).

Animal Research

All animal work in this thesis was carried out according to the Animals (Scientific Procedures) Act 1986 Amendment Regulations 2012 (SI 2012/3039) with approval from the University of Oxford and Imperial College London Ethical Review Committee (PPL 30/2889 Oxford, 70/8788 Imperial). The Office of Laboratory Animal Welfare Assurance for Imperial College covers all Public Health Service supported activities involving live vertebrates in the US (no. A5634-01). Rats and mice were kept in individually ventilated cages.

Murine Vaccination

Sporozoites were diluted to 30-40×10⁴ sporozoites/mL in Schneider's drosophila medium and 100 μL injected per mouse.

Falciparum Fluorescent Microscopy

P. falciparum-infected cells were imaged on a Nikon wide-field microscope. Image processing and analysis was automated by running a custom macro in Fiji. Quantification of intracellular versus extracellular parasites was determined using the macro. Cell infection was calculated as the % ratio of intracellular parasites to HC-04 nuclei.

Theileria parva Parasite Material

A single vial of 1 ml of homogenized Theileria parva-infected Rhipicephalus appendiculatus ticks was collected (approximately 5-10 coarsely ground R. appendiculatus adults), frozen and stored in liquid Nitrogen. The vial was thawed and centrifuged at 100 xg for 5 minutes to pellet large insoluble particular material (+P). A second high-speed centrifugation step followed at 14,000 rpm for 5 minutes yielding a supernatant fraction (+S) and pellet fraction presumed to contain Theileria sporozoites (+).

Free-Flow Electrophoresis for Theileria parva Separation

Prior to sporozoite extraction the FFE machine (FFE Service GmbH) was setup for continuous ZE (cZE) using a 0.5 mm ZE spacer. A separation buffer of 10 mM triethanolamine (TEA), 10 mM glacial acetic acid (HAc) and 250 mM sucrose was used with a stabilisation buffer of 100 mM TEA, 100 mM HAc and 250 mM sucrose injected into the separation chamber at 150-180 mL/hr. Electrodes were kept in 100 mM TEA, 100 mM HAc and 250 mM sucrose with a voltage of 900-950V and current and power limit of 150 mA and 150 W respectively. The pelleted parasites were resuspended in separation buffer and injected into the separation chamber at the cathode end at a rate of 700 μL/hr and fractions collected 14 min after injection started and stopped 14 min after sample finished. Fractions were collected in 2 mL protein lo-bind deep well plates (Eppendorf).

Dot Blot

Dot blots for each of 96-well fractions were collected and probed with either polyclonal serum (made by immunising a rat with 60 μg schizont protein suspension, isolated from TaCl2 cells(4)) at 1:2000 dilution or a mouse monoclonal antibody to T. parva Hsp70 protein (5) again at 1:2000 dilution (both antibodies a kind gift from Professor Philipp Olias, Institute of Animal Pathology, University of Bern, Switzerland). A secondary HRP-conjugated antibody was used at 1:500. Densitometry was performed using the ImageLab software (BioRad Laboratories).

Statistical Analysis

Data was assessed for normality and equality of variance and used to determine the suitable statistical test as per John Tukey's exploratory data analysis method (54). Parametric data was assessed using a t-test and non-parametrically using a Mann-Whitney U test for single treatment comparisons. Multiple treatments were compared using a t-test with Bonferroni correction. Kaplan-Meier curves were compared using the Mantel-Cox test.

Results

Rapid isolation of sporozoites from whole mosquitoes by density and charge Infection rates of manual salivary gland dissected (SGD)-sporozoites in in vitro cell cultures using HepG2 or primary hepatocytes achieved less than 1% infected cells, consistent with previous studies (FIG. 9). To explore alternative approaches that improved purity and infectivity of parasites, a combinatorial approach was taken that included whole mosquito homogenisation, filtration, Accudenz density gradient and FFE (FIG. 1a). —MalPure V1.0

MalPure 2.0 includes a combination of vital changes (i) removal of abdomens prior to homogenisation, (ii) addition of extra filters for size exclusion and optional differential centrifugation, (iii) the option of replacement of the density gradient with a sephadex column and (iv) use of new FFE protocol (iZE) (FIG. 1-2b). Some of these enhancements have also been added to MalPure V1.0 (FIG. 1-2a).

Homogenisation:

To overcome the limiting factors of sporozoite yield and reduce the time from isolation till addition to tissue culture, whole mosquitoes were homogenised to release mCherry-expressing P. berghei sporozoites. For MalPure V2.0 samples were homogenised using an automated system (gentleMACS) in specific tubes (FIG. 1-2e), in contrast to manual homogenisation with MalPure V1.0 (FIG. 1b). In some cases different parts of the mosquito were removed dependent on the maturity of sporozoites required. Alternatively samples could be homogenised by botoscilicate beads.

Filtration

Homogenate was filtered sequentially through 100 μm to 200 μm filters in as short a period of time as possible (approx. 10 min). Up to 1000 mosquitoes at once were processed using this protocol. —MalPure V1.0

Additionally, a 10 μm filter was added and optional differential centrifugation at 18×g to remove larger debris. The inventors note that this may mitigate need to abdominal removal (Differential centrifugation)—MalPure V2.0

Density Gradient Purification:

The filtered mosquito homogenate/mash (abbreviated to M) was separated by density centrifugation using Accudenz (abbreviated to MA when combined with M), as previously described (22), to remove larger mosquito-associated debris from sporozoites (FIG. 1b). —MalPure V1.0

Alternatively, sample could be pre-purfied using spehadex-G15 column (FIG. 1-2f). —MalPure V2.0

Continuous Zone Electrophoresis (MalPure V1.0):

The sporozoite layer was subsequently separated based on total net charge by FFE (total process abbreviated to MAF when combined with MA; FIG. 1c) using a continuous zone electrophoresis (cZE) mode with a sucrose-triethanolamine (TEA) buffer at physiological pH (see Methods). The 96 FFE output fractions were subsequently assessed by light microscopy or fluorescent plate reader for mCherry fluorescence (FIG. 1d, representative plots). Sporozoites showed a highly reproducible separation, independent of sporozoite dose injected, the majority of parasites separating into a single fraction with a characteristic tail that elongated as sporozoite dose load increased (FIG. 10). The peak fraction was used for all remaining experiments. FFE resulted in an average loss of yield of ˜30% from MA input, with approximately 600 mosquitoes processed in 2 hours. —MalPureV1.0

Interval Zone Electrophoresis (MalPure V2.0):

Alternatively, the pre-purified sporozoites were separated using an the iZE FFE method (FIG. 1-2c), which prevents samples reaching their isoelectric point. When using this method sporozoites separate into two peaks (FIG. 1-2d), the smaller peak contains highly pure sporozoites and is referred to as the ‘highly pure peak’.

MAF purified sporozoites show reduced contamination with mosquito-associated proteins and debris compared to manual salivary-gland dissected sporozoites Following separation, mosquito contaminants were assessed in comparison to those isolated by manual SGD. Samples were normalised in mosquito equivalents (meq), based on number of mosquitoes (mq) homogenised and volume (units: mq/mL) as opposed to sporozoite dose, which can vary between batches. To assess total protein contaminants, extract from uninfected mosquitoes and P. berghei infected mosquitoes were separated by FFE at three different mosquito equivalents (300, 100 and 50 mq/mL) following MA purification. Infected mosquitoes were used as controls to confirm the location of the peak FFE sporozoite fraction. All media used for protein assessment was protein free. Equivalent volumes were injected and collected for each treatment and total protein in each fraction quantified (FIG. 3 shows a). All peak sporozoite fractions had undetectable levels of protein in uninfected mosquito samples (assessed by bicinchoninic assay; BCA), indicating that protein contaminants had lower electrophoretic mobility than the sporozoite peak fraction. Protein contamination did however show a marked shift towards the sporozoite peak with higher mosquito dose.

Subsequently, to directly compare the different steps of the purification process each step was normalised to an equivalent meq dose (200 mq/mL) and 4 meq's worth run on a reducing SDS-PAGE and silver-stained (sensitivity of 25 ng) to assess protein levels. Uninfected dissected salivary gland homogenate was included to represent the current method. Comparing steps using uninfected mosquitoes it was evident that M and MA gave the highest total protein levels, followed by SGD preparations, with MAF showing almost undetectable levels of protein contaminants (FIG. 3 shows b). Levels of detectable protein contamination increased with increasing meq's applied to the FFE chamber. When compared to the M treatment, uninfected dissected salivary glands showed a 63.1% reduction in total protein levels, whilst uninfected MAF purified mosquitoes showed a 99.9, 100 and 100% reduction in detectable total protein for 300, 100 and 50 mq/mL respectively. A similar trend was seen when infected mosquitoes were used (FIG. 11a). Finally, a comparison between purification steps when infected dissected salivary glands (D) were used as the source for homogenisation instead of whole mosquitoes (M) (Dissected+Accudenz, DA; Dissected+Accudenz+FFE, DAF; Dissected+FFE, DF) showed a similar purification trend (FIG. 3 showsc left). To further illustrate our methods ability to remove free protein, each step (again normalised to 4 meq) was probed using a monoclonal antibody against one of the most abundant sporozoite surface protein CSP, revealing the complete removal of all detectable free CSP degradation products in FFE samples, which were abundant in dissected salivary gland homogenate (FIG. 3 shows c right). Moreover, the inventors did not detect mosquito actin in MAF purified sporozoites, another abundant contaminant in sporozoite samples obtained through SGD (FIG. 11b).

Whilst higher mosquito equivalents (100-300 mq/mL) used for FFE purification increased the amount of mosquito contaminants encroaching on the sporozoite fraction, the peak sporozoite fraction was still free of detectable protein. The location of the peak sporozoite fraction was, however, not affected by mosquito dose. For the remainder of this study, unless specified otherwise, FFE experiments were run using a meq dose of 100 mq/mL injected into the chamber.

To further explore the purity of FFE-derived samples, purification steps (normalised by meq) were pelleted by centrifugation, demonstrating a clear visual reduction in mosquito associated debris such as melanin from the cuticle (FIG. 3 shows d). Samples from SGD preparations and MAF were also diluted to the same concentration of sporozoites and visualised by brightfield microscopy (FIG. 3 shows e). Large pieces of debris, including a whole salivary gland were seen in the SGD treatment but not in MAF. The morphology of the sporozoites from each method (SGD Vs. MAF) was similar. Inspection of each step of purification from whole mosquito homogenate, normalised by sporozoite number (FIG. 3 shows f), further underscored the significant reduction in visible debris at each stage.

Finally, liquid chromatography tandem mass spectrometry (LC-MS/MS) analysis of each stage was used to sensitively assess protein purity at each stage of purification. LC-MS/MS raw data were searched against the Uniprot-Swissprot database using the MASCOT search algorithm. This led to the identification of several insect proteins across all three stages of purification (e.g. ATP synthase subunits a and b; Myosin heavy chain; ADP, ATP carrier protein 1/2) as well as other contaminating proteins (e.g. haemoglobin subunit b, specifically identified in MA). Importantly, P. berghei CSP protein was identified with three peptides exclusively in the fraction purified by MAF (FIG. 3 shows g), indicating that FFE-purified parasites were sufficiently enriched, with mosquito as well as other contaminating proteins depleted, to allow successful identification of this abundant sporozoite surface protein.

The Malpure 2.0 method shows increased protein purity as assessed by BCA, silver stain and pellets (FIG. 2-2a-c), even when injecting at higher mq/mL onto the machine. The output is visually clean of contaminants (FIG. 2-2d). —MalPure V2.0

Purification by FFE can Yield Bacterial Contamination-Free Sporozoites

Having developed a workflow for sporozoite isolation, the inventors next sought to test the aseptic condition of purified parasites. A serial dilution of samples normalised to equal meq from each stage of purification was grown in the non-selective medium, tryptic soya broth (TSB), which is suited to the growth of a large variety of aerobic bacteria (55). After growth for 16 hr at 37° C., absorbance was measured at 600 nm (OD600) (Figure a). Only MAF purified samples reported undetectable OD600 absorbance during the seven dilutions, with MA and SGD parasites showing similar absorbance levels. Bacterial contamination was also assessed by measuring bacteria colony forming units per mL (cfu/mL) on blood-agar plates (samples normalised by meq's; 200 mq/mL). Homogenates from whole uninfected mosquitoes (Figure b) had the highest bacteria load (M: 12.0 log cfu/mL), which was reduced by Accudenz centrifugation (MA: 7.0 log cfu/mL), but remained significantly higher than manual dissection (SGD: 6.1 log cfu/mL). The sporozoite peak fraction from FFE purification of MA injected at meq's of 300, 100 and 50 mq/mL caused a significant reduction of bacterial load compared to manual dissection (MAF: 300 mq/mL: 4.9 log cfu/mL; 100 mq/mL: 4.2 log cfu/mL; 50 mq/mL: 3.9 log cfu/mL). The lowest FFE injected dose saw the most significant drop of 2.2 log in bacterial load compared to SGD, which translates to a 173-fold reduction in the bacterial load added to a hepatocyte culture compared to sporozoites obtained by SGD. When mosquitoes were sporozoite infected, a similar trend was also observed, with MAF loaded at 100 mq/mL able to significantly reduce bacterial load by 1.3 log compared to SGD (Figure c). Interestingly, when dissected salivary glands, rather than whole mosquitoes were used as homogenate, Accudenz purification (DA) significantly reduced the bacterial load by 1.7 log compared to SGD and when salivary gland homogenate was separated directly by FFE without density gradient centrifugation (DF) a larger reduction of 2.4 log was observed. However, when complete purification was conducted on dissected salivary glands (DAF) the process was able to remove all bacteria detectable by blood-plate agar growth (Figure d). Thus, by combining Accudenz and FFE with SGD, sterile sporozoites could be obtained (FIG. 12). To facilitate further analysis, MAF purification using an FFE input of 100 mq/mL was used for the remainder of FFE experiments described.

Using MalPure 2.0, sporozoites can be purified aseptically without prior salivary gland dissection, instead, only removal of abdomens is required (FIG. 2-3).

MAF purified sporozoites show improved infectivity compared to SGD sporozoites Following assessment of sporozoite purification and sterility the effect of purification on parasite viability was assessed by a number of in vitro and in vivo techniques. The motility of sporozoites is commonly used to assess viability (17,27), therefore motility of MAF purified sporozoites were compared to SGD parasites by analysis of their static, attached waving or gliding (clockwise or counter clockwise) 2D motion (52) (Figure a-c). Mean velocity (Figure b) and overall percentage of motility (Figure c) showed no significant difference between SGD and MAF purified sporozoites in any of the movement states. Next, in vitro infection rates of MAF purified sporozoites were assessed in HepG2 hepatoma and primary rat hepatocytes by RT-PCR, microscopy and flow cytometry. RT-PCR analysis of the absolute copy number of the housekeeping gene P. berghei hsp70 by RT-PCR, normalised to 1000 hsp70 copies for the SGD treatment, showed a 1.5 and 2.1 times higher copy number in MAF sporozoite infected HepG2 and primary rat hepatocytes compared to SGD sporozoite infected hepatocytes, respectively (Figure d).

Additionally, 24 hr time-lapse videos were taken of primary rat hepatocytes after infection with sporozoites and invasion rate determined by counting settled exo-erythrocytic forms (EEFs) present at 24 hr. Infections with MAF sporozoites showed a markedly improved infection rate of 5.4% compared to 0.4% when using SGD sporozoites (Figure e). When assessed by flow cytometry, rat primary hepatocytes infected with MAF sporozoites showed infection rates of 10.37% (302 out of 2808 cells) (FIG. 13a). Complete liver-stage development of P. berghei MAF sporozoites was shown by the presence of liver-stage schizonts with anatomically normal morphology in fixed HepG2 hepatocytes at 52 hrs after addition of sporozoites (Figure f). The high infection rates are well-illustrated by full views of culture wells containing infected HepG2 hepatocytes at 52 hrs (FIG. 13b). These results show that FFE-based sporozoite purification increases the rate of in vitro infections substantially over manually dissected sporozoites, making them highly suitable for in vitro work, especially when their high purity is also considered, avoiding the effect of mosquito contaminants on assay readouts.

Subsequently, to explore in vivo infectivity and compare the outcome of different purification strategies, mice were challenged with sporozoites by intravenous (i.v.) injection and infectivity determined by measuring the time to reach 1% blood stage parasitaemia (prepatent period). Of note, since gliding motility of sporozoites in dissection buffer has been shown to decline over time (27), the inventors performed all isolation procedures in the same time period before injection into mice. Initially mice were infected intravenously with an escalating dose from 1000-5000 of MAF purified sporozoites, which all led to blood stage infection (Figure g). Furthermore, mice infected with 5000 sporozoites purified by either MA, MAF or SGD sporozoites all became blood stage positive (Figure h), but the time to 1% parasitaemia was on average 0.66 and 0.59 days longer for MA- and MAF-infected mice, respectively, than for mice injected with SGD sporozoites (MA**p=0.0049, MAF**p=0.0031; Mantel-Cox Test). There was, however, no significant difference between MAF or MA.

The modest reduction in in vivo infectivity of MA and MAF sporozoites compared to SGD sporozoites suggested that either Accudenz or isolation of sporozoites from whole mosquitoes may cause a reduction in infectiousness. To address this, mice were subsequently i.v. injected with 5000 sporozoites purified by MA using different mosquito homogenate sources (whole, decapitated, abdomen removed) and infectivity was compared to SGD sporozoites by assessing the time to reach 1% parasitaemia (Figure i). Removal of the abdomen prior to homogenisation was sufficient to revert the delay in parasitaemia, unlike using whole mosquitoes or removal of the head, which still caused a significant delay (MA-Whole**p=0.0016, MA-No Head**p=0.0016; Mantel-Cox Test). Increasing the dose of whole mosquito origin MA mosquitoes (180%, 1.8-fold increase) caused a reduction in delay indicating an increase in infectious dose. These results suggest that removal of immature sporozoite in oocysts (and possibly haemolymph) (56) found in the abdomen avoids the delay in time to patency. To investigate the contribution of oocyst-derived/haemolymph sporozoites, mosquitoes at 21 days post infectious blood feed were separated into abdomen, thorax (containing the salivary glands) and head. These different parts contained 70%, 29% and 1% of total sporozoites respectively (Figure j).

To confirm the delay was due to abdominal immature sporozoites, mice were infected with MAF purified sporozoites from mosquitoes that had their abdomens removed (MaAF; Figure k). Mice infected with 1000 MaAF purified sporozoites showed a significant decrease (*p=0.0415; Mantel-Cox Test) in time to 1% blood parasitaemia compared to mice infected with the same number of SGD sporozoites. Noteworthily, the same was not observed when only MA (i.e. no FFE) without abdomens (MA-No Abdomen) were used (Figure i). Combined, our observations indicate that MAF is thus also capable of significantly increasing the in vivo infectivity of sporozoites. The MAF method therefore provides a flexible workflow for the purification of high purity sporozoites which can be obtained from different sources (i.e. whole mosquitoes, no abdomens, no thorax/head or dissected salivary glands) as suited to the purpose, whilst giving superior infection rates in vitro and in vivo.

With MalPure 2.0 the inventors also see an increase in the fitness of parasites (i.e. their ability to establish a blood-stage infection in mice), which is clear at lower infective doses (FIG. 4-2a-b). Suggesting that for a vaccine a lower dose of MalPure sporozoites would be required,

Additionally, P. falciparum sporozoites can be successfully purified by MalPure V1.0 and V2.0 to successfully invade hepatocytes in vitro (FIG. 4-2c).

Purified MAF Human P. falciparum Sporozoites are More Infective In Vitro Compared to SGD

With P. falciparum, MAF sporozoites increased HC-04 invasions by 2.98-fold, with a 3-fold increase in the percentage of sporozoites inside host cells compared to SGD sporozoites (FIG. 16a-b).

Purified MAF Human P. Falciparum Sporozoites Offer Sterile Protection as an Irradiated Parasite Vaccine

Having developed a method which produces sporozoites with a high purity and improved infectivity over SGD sporozoites the inventors next sought to assess the potential of the MAF sporozoites as a radiation attenuated sporozoite vaccine (RASv). Prior to immunisation the effective irradiation dose was determined to be 60Gy by i.v. challenge with varying doses of gamma irradiated sporozoites (FIG. 17a). Mice were immunised i.v. using a three-immunisation regime with 40,000 irradiated sporozoites, two weeks apart. In parallel, control mice were immunised with plain medium as controls (FIG. 17b). Immunisation with MAF sporozoites i.v. achieved complete protection against both P. berghei and P. falciparum (FIG. 17c-d). This was at least comparable to irradiated SGD sporozoites. The inventors next sought to test the efficacy of the MAF sporozoites when given intramuscularly (i.m.) with AddaVax adjuvant, a more practical route to administration. The inventor's sporozoites showed a significant 70% and 67% efficacy for P. berghei and P. falciparum respectively.

Application Offree-Flow Electrophoresis to Purification of Theileria parva Sporozoites from Rhipicephalus appendiculatus Tick-Infected Homogenate

Processing R. appendiculatus tick homogenate infected with T. parva by centrifugation and free-flow electrophoresis (FFE) resulted in a clear fraction of purified T. parva parasites. Two alternative methods were trialled (A, 900 volts at 150 mL/hour vs. B 950 volts at 180 mL/hour). As predicted method A (slower and lower voltage) produced a narrower fraction peak as determined by densitometry of dot blots than method B (FIG. 18). This demonstrates clearly the first steps towards defining a set of experimental conditions that can isolate purified Theileria sporozoites away from infected ticks.

### DISCUSSION

Although the pre-erythrocytic stage of malaria is an attractive intervention point to prevent the development of clinical malaria in human hosts, the field has struggled to understand and identify possible intervention targets of this developmental stage (57). A major barrier to progress has been the limited success of in vitro liver stage systems that yield low numbers of infected hepatocytes (10, 13, 16, 58, 59) when compared to comparable asexual blood-stage or sexual stage high throughput in vitro platforms (60-63). One of the key stumbling blocks for in vitro cultures is obtaining large numbers of purified and highly hepatocyte-infectious sporozoites. The work presented here helps to break down this major barrier to liver stage research, showing the development of a protocol for the purification of sporozoites in large quantities in a short period of time that is associated with markedly improved infection rates when compared to industry-standard salivary gland dissection.

Importantly, in addition to the speed and infectivity of sporozoites derived by FFE, parasites purified through this process yield entirely sterile sporozoites. This is especially important for controlling bacterial growth in long-term cultures. Sporozoite hepatocyte cultures, in particular those from P. falciparum, are commonly associated with contamination because they require at least seven days to complete the full developmental cycle. In addition to bacterial contamination, isolation of SGD sporozoites, but not MAF purified sporozoites, likely brings with it other host factors that may act as hepatocyte stress factors as evidence by the development of abnormal morphology in primary hepatocytes (FIG. 14). Only recently has it been shown that mosquito salivary proteins are able to modify the host immune response (31). Furthermore, recent work by Billman and colleagues (64) show that dissected sporozoites without purification by Accudenz density centrifugation (which the inventors show can cause a modest reduction in bacterial and total protein loads when using dissected salivary glands; FIG. 2c, FIG. 3d) caused a reduction in pre-primed sporozoite (boost) specific CD8 T-cell responses compared to Accudenz, something undesirable for an effective sporozoite vaccine. High levels of mosquito contaminants may also cause innate immune upregulation in vivo with unknown, if not confounding, effects on vaccination studies. Critically, in our hands, MAF purification led to an almost undetectable level of contaminating mosquito proteins by silver stain, which detects a minimum of 0.25 ng of protein. Whilst total amount of mosquito homogenate input was a key variable for this, if dissections are a viable option, DAF could be used to avoid the use of antibiotics in mosquito rearing and subsequent hepatocyte cultures, the effect of which on parasite development is currently unknown.

One notable difference observed between MAF-derived versus SGD sporozoites was the integrity of the major sporozoite surface protein, CSP. Immunoblotting of each separation stage against CSP highlighted three phenomena. Firstly, there was a reduction in total CSP following each step, indicating a loss of sporozoites and/or CSP shedding at each stage. Secondly, steps prior to FFE separation contained CSP fragments (as detected with anti-CSP antibody 3D11 (53) (FIG. 2c) likely derived from breakdown products containing central repeats within the CSP protein (65). FFE would therefore be predicted to effectively remove free and degraded protein away from whole parasites such as shown for the mosquito actin protein (Supplementary FIG. 3b). This may be critical for infection outcomes of MAF versus SGD derived sporozoites. Thirdly, CSP is expressed on the surface of sporozoites and during hepatocyte infection it becomes proteolytically cleaved, triggered by HSPGs on the hepatocyte surface and therefore dependent on hepatocyte contact (66,67). Following MAF purification the ratio of uncleaved:cleaved was 1:7.8, compared to 1:1.8 for SGD sporozoites (assessed by densitometry), indicating that MAF purified sporozoites have >4 times more cleaved CSP on their surface. This is of interest since approximately 80% of the sporozoites from a whole mosquito are found in the abdomen (i.e. less mature). Genetically modified sporozoites which express the cleaved form invade more hepatocytes in vitro (14). This may therefore be another critical molecular factor contributing to improved infectivity seen with MAF. In addition to this, a recent study has shown that a mosquito salivary protein, mosGILT, negatively modulated sporozoite motility (30), suggesting other mosquito-associated factors that reduce hepatocyte infectivity may still be forthcoming and are likely to be purified away from sporozoites using the MAF protocol, improving hepatocyte infectivity.

Using MAF sporozoites for in vitro infections led to a significant increase in successful infections from 24 to 40 hr after invasion, measured by either RT-PCR or microscopy. Infection rates of 10.4% were observed by flow cytometry. These sporozoites fully developed into late-stage exoerythrocytic schizonts in vitro (FIG. 4f) and ex vivo (FIG. 13). Whilst using MAF sporozoites from whole mosquitoes led to a reduction in in vivo infectivity in mice, removing only the abdomen prior to homogenisation enhanced infectivity over SGD. A possible explanation for the decrease in in vivo infectiousness with whole mosquito derived sporozoites may relate to the sporozoite forms being injected. At day 20 post infection of mosquitoes, Coppi and colleagues (14) reported that 54% of sporozoites were found in salivary glands, 18% in the haemolymph and 28% associated with the midgut. At day 21, the inventors found 80% of sporozoites in the abdomen. Studies on the infectiousness of each of these sporozoite stages has shown that oocyst sporozoites (i.e. within the abdominal section) are over 1000-fold less infectious than salivary gland sporozoites by intravenous challenge (56). Thus, a significant proportion of injected sporozoites post-isolation may potentially be poorly infective. These poorly infective oocyst sporozoites show very limited gliding motility (17, 52, 56) which improves through development into haemolymph and finally salivary gland sporozoites, which have greater than 80% gliding motility (17,52). Related to this, haemolymph sporozoites have shown variable in vivo infectivity in studies which in some cases is comparable to SGD sporozoites (17,56). As might be expected, the requirement for a motile phenotype is critical for migration from skin to the perisinusoidal space in the liver (1). In our study there was no significant difference in motility between whole mosquito MAF sporozoites and SGD sporozoites in contradiction to these previous studies (17,52). A possible explanation for this could be the removal of mosquito associated proteins by our purification protocol which may be inhibiting motility. This improved motility may also contribute to the improved infectivity.

Overall, the flexibility of our protocol, when combined with its high yield, high purity, improved infectivity and scalability enable a wealth of applications, from small scale in vitro experiments to mass throughput drug screening assays and significantly enhanced whole-parasite vaccine opportunities. For example, many hundreds of whole mosquitoes can be directly processed to provide tens of millions of sporozoites in hours (the inventors have validated up to 1000 mosquitoes in one purification). By way of illustration, in one of our experiments rats were i.v. challenged with 30×10⁷ MAF purified P. berghei GFP sporozoites from 400 mosquitoes within 2 hours (FIG. 15). A recent high throughput drug screening assay of the liver-stage required over 2 years of labour-intensive SGD's from over a million mosquitoes to obtain enough sporozoites for the screening (68). Sporozoites from a similar number of mosquitoes could have been harvested within a few months using our approach. Alternatively, if in vivo viability is important the abdomens can be removed first, a simple task requiring little skill or time (unlike SGD). Finally, supporting the emerging interest in how sporozoites colonise the mosquito salivary glands (69,70), our protocol allows the flexibility of using an input of mosquitoes post head/thorax removal prior to homogenisation to isolate midgut derived sporozoites only.

In conclusion, the work presented here shows the development of a complete protocol for purification of large numbers of highly infectious sporozoites in rapid and scalable approach that is entirely compatible with basic biological, drug-screening and whole-parasite vaccine studies. Based on FFE, our approach yields sporozoites at higher purity compared to those from dissected preparations alone and is associated with a marked increase in in vitro hepatocyte infections and enhanced in vivo infectivity when abdominal sporozoites are removed. Sporozoites harvested by FFE also show markedly reduced levels of bacterial and when combined with SGD, sporozoites isolated using FFE can be routinely produced entirely bacteria-free. Importantly, the procedure permits purification of high numbers of sporozoites in a very short period of time, promising a scalable means to improve a diversity of studies. For basic sciences, demonstration of the presence of CSP peptides provides evidence that this method will be an important step towards single cell-omic studies that will require large amounts of highly pure sporozoites, free of the mosquito-associated contaminants that often limit our ability to draw conclusions from these studies (71-73). The inventors believe that application of this method will have significant implications for increasing our understanding of malaria liver stage biology and the development of therapeutic approaches that thwart it.

The inventors work in Theileria species shows that the methods developed herein to purify Plasmodium species parasites is also suitable for purifying other obligate parasitic species, such as Theileria parva. Since mosquito proteins are known to negatively impact on Plasmodium parasite infection and transmission from mosquito to human as well as impact immunity that arises (79, 80) the inventors anticipate that the separation of Theileria sporozoites away from tick material, and its further optimisation towards purity of aseptic parasites, as shown in FIG. 18, will provide a significant boost to infection and treatment method vaccine efficacy and potential for major cost savings via automation of the process.

### REFERENCES

1. Prudêncio M, Rodriguez A, Mota M M. The silent path to thousands of merozoites: the Plasmodium liver stage. Nature Reviews Microbiology. 2006 November; 4(11):849-56.
2. Vanderberg J P. Plasmodium berghei: quantitation of sporozoites injected by mosquitoes feeding on a rodent host. Exp Parasitol. 1977 June; 42(1):169-81.
3. Sigler C I, Leland P, Hollingdale M R. In vitro infectivity of irradiated Plasmodium berghei sporozoites to cultured hepatoma cells. Am J Trop Med Hyg. 1984 July; 33(4):544-7.
4. Ntssler A, Follezou J Y, Miltgen F, Mazier D. Effect of irradiation on Plasmodium sporozoites depends on the species of hepatocyte infected. Trop Med Parasitol. 1989 December; 40(4):468-9.
5. Hoffman S L, Billingsley P F, James E, Richman A, Loyevsky M, Li T, et al. Development of a metabolically active, non-replicating sporozoite vaccine to prevent Plasmodium falciparum malaria. Human Vaccines. Taylor & Francis; 2010 Jan. 1; 6(1):97-106.
6. Hoffman S L, Doolan D L. Malaria vaccines-targeting infected hepatocytes—Nature Medicine. Nat Med. 2000 Nov. 1; 6(11):1218-9.
7. Hoffman S L, Goh L M L, Luke T C, Schneider I, Le T P, Doolan D L, et al. Protection of Humans against Malaria by Immunization with Radiation-Attenuated Plasmodium falciparumSporozoites. J INFECT DIS. 2002 Apr. 15; 185(8):1155-64.
8. HAWKING F. Pre-erythrocytic Stage in Mammalian Malaria Parasites. Nature. 1948 Jan. 31; 161(4083):175-5.
9. Hollingdale M R, Leland P, Schwartz A L. In vitro cultivation of the exoerythrocytic stage of Plasmodium berghei in a hepatoma cell line. Am J Trop Med Hyg. 1983 July; 32(4):682-4.
10. Sattabongkot J, Yimamnuaychoke N, Leelaudomlipi S, Rasameesoraj M, Jenwithisuk R, Coleman R E, et al. Establishment of a human hepatocyte line that supports in vitro development of the exo-erythrocytic stages of the malaria parasites Plasmodium falciparum and P. vivax. Am J Trop Med Hyg. 2006 May; 74(5):708-15.
11. March S, Ng S, Velmurugan S, Galstian A, Shan J, Logan D J, et al. A Microscale Human Liver Platform that Supports the Hepatic Stages of Plasmodium falciparum and vivax. Cell Host Microbe. 2013 July; 14(1):104-15.
12. Gosling R, Seidlein von L. The Future of the RTS,S/ASo1 Malaria Vaccine: An Alternative Development Plan. PLoS Med. Public Library of Science; 2016 Apr. 12; 13(4):e1001994.
13. Kaiser K, Camargo N, Kappe S H I. Transformation of Sporozoites into Early Exoerythrocytic Malaria Parasites Does Not Require Host Cells. The Rockefeller University Press; 2003 Apr. 21; 197(8):1045-50.
14. Coppi A, Natarajan R, Pradel G, Bennett B L, James E R, Roggero M A, et al. The malaria circumsporozoite protein has two functional domains, each with distinct roles as sporozoites journey from mosquito to mammalian host. Rockefeller University Press; 2011 Feb. 14; 208(2):341-56.
15. Zou X, House B L, Zyzak M D, Richie T L, Gerbasi V R. Towards an optimized inhibition of liver stage development assay (ILSDA) for Plasmodium falciparum. Malar J. 2013; 12(1):394.
16. House B L, Hollingdale M R, Sacci J B Jr., Richie T L. Functional immunoassays using an in-vitro malaria liver-stage infection model: where do we go from here? Trends Parasitol. 2009 November; 25(11):525-33.
17. Sato Y, Montagna G N, Matuschewski K. Plasmodium berghei sporozoites acquire virulence and immunogenicity during mosquito hemocoel transit. Infect Immun. American Society for Microbiology; 2014 March; 82(3):1164-72.
18. Ishino T, Yano K, Chinzei Y, Yuda M. Cell-Passage Activity Is Required for the Malarial Parasite to Cross the Liver Sinusoidal Cell Layer. Gary Ward, editor. PLoS Biol. Public Library of Science; 2004 Jan. 20; 2(1):e4.
19. Ishino T, Chinzei Y, Yuda M. Two proteins with 6-cys motifs are required for malarial parasites to commit to infection of the hepatocyte. Molecular Microbiology. 2005 Sep. 5; 58(5):1264-75.
20. Kariu T, Ishino T, Yano K, Chinzei Y, Yuda M. CelTOS, a novel malarial protein that mediates transmission to mosquito and vertebrate hosts. Molecular Microbiology. 2006 Jan. 20; 59(5):1369-79.
21. Hollingdale M R, Nardin E H, Tharavanij S, Schwartz A L, Nussenzweig R S. Inhibition of entry of Plasmodium falciparum and P. vivax sporozoites into cultured cells; an in vitro assay of protective antibodies. The Journal of Immunology. American Association of Immunologists; 1984 February; 132(2):909-13.
22. Kennedy M, Fishbaugher M E, Vaughan A M, Patrapuvich R, Boonhok R, Yimamnuaychok N, et al. A rapid and scalable density gradient purification method for Plasmodium sporozoites. Malar J. BioMed Central Ltd; 2012 Dec. 17; 11(1):421.
23. Mazier D, Beaudoin R L, Mellouk S, Druilhe P, Texier B, Trosper J, et al. Complete development of hepatic stages of Plasmodium falciparum in vitro. Science. 1985 Jan. 25; 227(4685):440-2.
24. Siau A, Silvie O, Franetich J-F, Yalaoui S, Marinach C, Hannoun L, et al. Temperature shift and host cell contact up-regulate sporozoite expression of Plasmodium falciparum genes involved in hepatocyte infection. Dietsch K, editor. PLoS Pathog. 2008 Aug. 8; 4(8):e1000121.
25. Corradetti A, Verolini F, Sebastiani A, Proietti A M, Amati L. FLUORESCENT ANTIBODY TESTING WITH SPOROZOITES OF PLASMODIA. Bulletin of the World Health Organization. World Health Organization; 1964; 30(5):747-50.
26. Bhanot P, Schauer K, Coppens I, Nussenzweig V. A Surface Phospholipase Is Involved in the Migration of Plasmodium Sporozoites through Cells. J Biol Chem. 2005 Feb. 18; 280(8):6752-60.
27. Lupton E J, Roth A, Patrapuvich R, Maher S P, Singh N, Sattabongkot J, et al. Enhancing longevity of Plasmodium vivax and P. falciparum sporozoites after dissection from mosquito salivary glands. Parasitology International. 2015 April; 64(2):211-8.
28. Hovlid M L, Winzeler E A. Phenotypic Screens in Antimalarial Drug Discovery. Trends Parasitol. Elsevier; 2016 Sep. 1; 32(9):697-707.
29. Ménard R, Tavares J, Cockburn I, Markus M, Zavala F, Amino R. Looking under the skin: the first steps in malarial infection and immunity. Nature Reviews Microbiology. Nature Research; 2013 Oct. 1; 11(10):701-12.
30. Schleicher T R, Yang J, Freudzon M, Rembisz A, Craft S, Hamilton M, et al. A mosquito salivary gland protein partially inhibits Plasmodium sporozoite cell traversal and transmission. Nature Communications. Nature Publishing Group; 2018 Jul. 25; 9(1):2908.
31. Vogt M B, Lahon A, Arya R P, Kneubehl A R, Clinton J L S, Paust S, et al. Mosquito saliva alone has profound effects on the human immune system. Dinglasan R R, editor. PLoS Negl Trop Dis. Public Library of Science; 2018 May 17; 12(5):e0006439.
32. Ozaki L S, Gwadz R W, Godson G N. Simple Centrifugation Method for Rapid Separation of Sporozoites from Mosquitoes. The Journal of Parasitology. 1984 October; 70(5):831.
33. Bosworth A B, Schneider I, Freier J E. Mass Isolation of Anopheles stephensi Salivary Glands Infected with Malarial Sporozoites. The Journal of Parasitology. 1975 August; 61(4):769.
34. Touray M G, Warburg A, Laughinghouse A, Krettli A U, Miller L H. Developmentally regulated infectivity of malaria sporozoites for mosquito salivary glands and the vertebrate host. 1992 Jun. 1; 175(6):1607-12.
35. KRETTLI A, CHEN DH, NUSSENZWEIG R S. Immunogenicity and Infectivity of Sporozoites of Mammalian Malaria Isolated by Density-Gradient Centrifugation. Journal of Eukaryotic Microbiology. Blackwell Publishing Ltd; 1973 Nov. 1; 20(5):662-5.
36. Beaudoin R L, Strome C P A, Mitchell F, Tubergen T A. Plasmodium berghei: Immunization of mice against the ANKA strain using the unaltered sporozoite as an antigen. Exp Parasitol. 1977 June; 42(1):1-s.
37. Pacheco N D, Strome C P A, Mitchell F, Bawden M P, Beaudoin R L. Rapid, Large-Scale Isolation of Plasmodium berghei Sporozoites from Infected Mosquitoes. The Journal of Parasitology. 1979 June; 65(3):414.
38. Wood D E, Smrkovski L L, McConnell E, Pacheco N D, Bawden M P. The use of membrane screen filters in the isolation of Plasmodium berghei sporozoites from mosquitos. Bulletin of the World Health Organization. World Health Organization; 1979; 57 Supp11(Suppl):69-74.
39. Schulman S, Oppenheim J D, Vanderberg J P. Plasmodium berghei and Plasmodium knowlesi: Serum binding to sporozoites. Exp Parasitol. 1980 June; 49(3):420-9.
40. Heidrich H-G, Danforth H D, Leef J L, Beaudoin R L. Free-Flow Electrophoretic Separation of Plasmodium berghei Sporozoites. The Journal of Parasitology. 1983 April; 69(2):360.
41. Mack S R, Vanderberg J P, Nawrot R. Column separation of Plasmodium berghei sporozoites. The Journal of Parasitology. 1978 February; 64(1):166-8.
42. MOSER G, BROHN FH, Danforth H D, NUSSENZWEIG R S. Sporozoites of Rodent and Simian Malaria, Purified by Anion Exchangers, Retain their Immunogenicity and Infectivity. Journal of Eukaryotic Microbiology. Blackwell Publishing Ltd; 1978 Feb. 1; 25(1):119-24.
43. Hannig K, Heidrich H-G. Free-Flow Electrophoresis: An Important Preparative and Analytical Technique for Biology, Biochemistry and Diagnostics. 1st ed. Darmstadt: Git Verlag; 1990.
44. Angrisano F, Riglar D T, Sturm A, Volz J C, Delves M J, Zuccala E S, et al. Spatial Localisation of Actin Filaments across Developmental Stages of the Malaria Parasite. Templeton T J, editor. PLoS ONE. Public Library of Science; 2012 February 28; 7(2):e32188.
45. Lin J-W, Annoura T, Sajid M, Chevalley-Maurel S, Ramesar J, Klop O, et al. A novel “gene insertion/marker out” (GIMO) method for transgene expression and gene complementation in rodent malaria parasites. Kappe S, editor. PLoS ONE. 2011; 6(12):e29289.
46. S Hopp C, Chiou K, RT Ragheb D, M Salman A, M Khan S, J Liu A, et al. Longitudinal analysis of Plasmodium sporozoite motility in the dermis reveals component of blood vessel recognition. eLife. 2014; 4.
47. Mueller A-K, Camargo N, Kaiser K, Andorfer C, Frevert U, Matuschewski K, et al. Plasmodium liver stage developmental arrest by depletion of a protein at the parasite-host interface. Proc Natl Acad Sci USA. National Academy of Sciences; 2005 Feb. 22; 102(8):3022-7.
48. Salman A M, Mogollon C M, Lin J-W, van Pul F J A, Janse C J, Khan S M. Generation of Transgenic Rodent Malaria Parasites Expressing Human Malaria Parasite Proteins. In: Malaria Vaccines. New York, N.Y.: Humana Press, New York, N.Y.; 2015. pp. 257-86. (Methods in Molecular Biology; vol. 1325).
49. Janse C J, Ramesar J, Waters A P. High-efficiency transfection and drug selection of genetically transformed blood stages of the rodent malaria parasite <i>Plasmodium berghei</i>. Nature Protocols 2006 1:1. Nature Publishing Group; 2006 Jun. 1; 1(1):346-56.
50. Ingber D E. Fibronectin controls capillary endothelial cell growth by modulating cell shape. Proc Natl Acad Sci USA. 1990 May; 87(9):3579-83.
51. Kostadinova R, Boess F, Applegate D, Suter L, Weiser T, Singer T, et al. A long-term three dimensional liver co-culture system for improved prediction of clinically relevant drug-induced hepatotoxicity. Toxicology and Applied Pharmacology. 2013 Apr. 1; 268(1):1-16.
52. Hegge S, Kudryashev M, Smith A, Frischknecht F. Automated classification of Plasmodium sporozoite movement patterns reveals a shift towards productive motility during salivary gland infection. Biotechnology Journal. WILEY-VCH Verlag; 2009 Jun. 1; 4(6):903-13.
53. Yoshida N, Nussenzweig R S, Potocnjak P, Nussenzweig V, Aikawa M. Hybridoma produces protective antibodies directed against the sporozoite stage of malaria parasite. Science. American Association for the Advancement of Science; 1980 Jan. 4; 207(4426):71-3.
54. Tukey J W. Exploratory Data Analysis. 1st ed. Pearson; 1977.
55. Winn W C, Koneman E W. Koneman's Color Atlas and Textbook of Diagnostic Microbiology. 7 ed. Lippincott Williams & Wilkins; 2016.
56. Vanderberg J P. Development of Infectivity by the Plasmodium berghei Sporozoite. The Journal of Parasitology. 1975 February; 61(1):43.
57. Theander T G, Lusingu J. Efficacy and safety of RTS, S/AS01 malaria vaccine with or without a booster dose in infants and children in Africa. Lancet. 2015.
58. Vaccines TMCGO. A Research Agenda for Malaria Eradication: Vaccines. PLoS Med. Public Library of Science; 2011 Jan. 25; 8(1):e1000398.
59. Drugs TMCGO. A Research Agenda for Malaria Eradication: Drugs. PLoS Med. Public Library of Science; 2011 Jan. 25; 8(1):e1000402.
60. Plouffe D, Brinker A, McNamara C, Henson K, Kato N, Kuhen K, et al. In silicoactivity profiling reveals the mechanism of action of antimalarials discovered in a high-throughput screen. Proc Natl Acad Sci USA. National Acad Sciences; 2008 Jun. 25; 105(26):9059-64.
61. O'Neill P M, Amewu R K, Charman S A, Sabbani S, Gnidig N F, Straimer J, et al. A tetraoxane-based antimalarial drug candidate that overcomes PfK13-C580Y dependent artemisinin resistance. Nature Communications. Nature Publishing Group; 2017 May 24; 8:ncomms15159.
62. Rottmann M, McNamara C, Yeung B K S, Lee M C S, Bin Zou, Russell B, et al. Spiroindolones, a Potent Compound Class for the Treatment of Malaria. Science. American Association for the Advancement of Science; 2010 Sep. 3; 329(5996):1175-80.
63. Ruecker A, Mathias D K, Straschil U, Churcher T S, Dinglasan R R, Leroy D, et al. A male and female gametocyte functional viability assay to identify biologically relevant malaria transmission-blocking drugs. Antimicrobial Agents and Chemotherapy. American Society for Microbiology Journals; 2014 Sep. 29; 58(12):AAC.03666-14-7302.
64. Billman Z P, Seilie A M, Murphy S C. Purification of Plasmodium Sporozoites Enhances Parasite-Specific C D8+ T Cell Responses. Adams J H, editor. Infect Immun. American Society for Microbiology; 2016 Aug. 1; 84(8):2233-42.
65. Ferguson D J P, Balaban A E, Patzewitz E-M, Wall R J, Hopp C S, Poulin B, et al. The Repeat Region of the Circumsporozoite Protein is Critical for Sporozoite Formation and Maturation in Plasmodium. Silvie O, editor. PLoS ONE. 2014 Dec. 1; 9(12):e113923.
66. Coppi A, Tewari R, Bishop J R, Bennett B L, Lawrence R, Esko J D, et al. Heparan sulfate proteoglycans provide a signal to Plasmodium sporozoites to stop migrating and productively invade host cells. —PubMed—NCBI. Cell Host Microbe. 2007 November; 2(5):316-27.
67. Coppi A, Pinzon-Ortiz C, Hutter C, Sinnis P. The Plasmodium circumsporozoite protein is proteolytically processed during cell invasion. Rockefeller University Press; 2005 Jan. 3; 201(1):27-33.
68. Antonova-Koch Y, Meister S, Abraham M, Luth M R, Ottilie S, Lukens A K, et al. Open-source discovery of chemical leads for next-generation chemoprotective antimalarials. Science. American Association for the Advancement of Science; 2018 Dec. 7; 362(6419):eaat9446.
69. O'Brochta D A, Alford R, Harrell R, Aluvihare C, Eappen A G, Li T, et al. Is Saglin a mosquito salivary gland receptor for Plasmodium falciparum? Malar J. 2019 Jan. 3; 18(1):1330.
70. Ishino T, Murata E, Tokunaga N, Baba M, Tachibana M, Thongkukiatkul A, et al. Rhoptry neck protein 2 expressed in Plasmodium sporozoites plays a crucial role during invasion of mosquito salivary glands. Cell Microbiol. John Wiley & Sons, Ltd (10.1111); 2019 Jan. 1; 21(1):e12964.
71. Carlton J M, Angiuoli S V, Suh B B, Kooij T W, Pertea M, Silva J C, et al. Genome sequence and comparative analysis of the model rodent malaria parasite Plasmodium yoelii yoelii. Nature. 2002 Oct. 3; 419(6906):512-9.
72. Florens L, Washburn M P, Raine J D, Anthony R M, Grainger M, Haynes J D, et al. A proteomic view of the Plasmodium falciparum life cycle. Nature. 2002 Oct. 3; 419(6906):520-6.
73. Lasonder E, Janse C J, van Gemert G-J, Mair G R, Vermunt A M W, Douradinha B G, et al. Proteomic Profiling of Plasmodium Sporozoite Maturation Identifies New Proteins Essential for Parasite Development and Infectivity. Goldberg D E, editor. PLoS Pathog. Public Library of Science; 2008 Oct. 31; 4(10):e1000195.
74. V. Nene et al., The biology of Theileria parva and control of East Coast fever—Current status and future trends. Ticks Tick Borne Dis 7, 549-564 (2016).
75. D. E. Radley et al., East coast fever: 3. Chemoprophylactic immunization of cattle using oxytetracycline and a combination of theilerial strains. Veterinary Parasitology 1, 51-60 (1975).
76. G. Lynen et al., East Coast fever immunisation field trial in crossbred dairy cattle in Hanang and Handeni districts in northern Tanzania. Trop Anim Health Prod 44, 567-572 (2012).
77. O. Wiens et al., Cell cycle-dependent phosphorylation of Theileria annulata schizont surface proteins. PLoS One 9, e103821 (2014).
78. C. Daubenberger et al., Molecular characterisation of a cognate 70 kDa heat shock protein of the protozoan Theileria parva. Mol Biochem Parasitol 85, 265-269 (1997).
79. T. R. Schleicher et al., A mosquito salivary gland protein partially inhibits Plasmodium sporozoite cell traversal and 50 transmission. Nat Commun 9, 2908 (2018).
80. M. B. Vogt et al., Mosquito saliva alone has profound effects on the human immune system. PLoS Negl Trop Dis 12, eooo6439 (2018).

1. A method for purifying a metabolically active obligate parasite of vertebrates and arthropods, the method comprising:
(A) providing an arthropod infected with an obligate parasite of vertebrates and arthropods;
(B) a) (i) homogenising the arthropod of step (A); and
(ii) filtering the homogenate of step (i); and/or
(iii) pre-purifying the filtered homogenate resulting from step (ii); or
b) (i) dissecting the salivary gland of the infected arthropod of step (A); and
(ii) homogenising the dissected glands from step (i);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

2. The method according to claim 1, comprising:
(A) providing an arthropod infected with an obligate parasite of vertebrates and arthropods;
(B) (b) (i) dissecting the salivary gland of the infected arthropod of step (A); and
(ii) homogenizing the dissected glands from step (i);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

3. The method according to claim 2, wherein step (B) further comprises:
(iii) filtering the homogenate of step (ii); and/or
(iv) pre-purifying the filtered homogenate resulting from step (iii).

4. The method according to claim 1, comprising:
(A) providing an arthropod infected with an obligate parasite of vertebrates and arthropods;
(B) (a) (i) homogenising the arthropod of step (A);
(ii) filtering the homogenate of step (i); and
(iii) pre-purifying the filtered homogenate resulting from step (ii);
(C) separating the homogenate resulting from step (B) into fractions by electrophoresis; and
(D) obtaining a fraction from step (C) comprising the obligate parasite of vertebrates and arthropods, thereby obtaining a purified metabolically active obligate parasite of vertebrates and arthropods.

5. The method according to any preceding claim, wherein the obligate parasite is a parasite selected from the group consisting of: Plasmodium species parasite, Theileria species parasite, Trypanosoma species parasite, Leishmania species parasite and protozoan parasites of arthropods and vector borne viruses.

6. The method according to any preceding claim, wherein the obligate parasite is a Plasmodium species parasite, optionally wherein the obligate parasite is P. falciparum.

7. The method according to any one of claims 1 to 5, wherein the obligate parasite is a Theileria species parasite, optionally wherein the obligate parasite is T. parva.

8. The method according to any preceding claim, wherein the arthropod is an arthropod selected from the group consisting of: a mosquito, Rhipicephalus spp., Tsetse fly and triatominae spp.

9. The method according to any one of claims 1 to 6 or claim 8, wherein the arthropod is a mosquito, optionally wherein the mosquito is selected from a group consisting of: Anopheles gambiaes; Anopheles coluzzi; Anopheles merus; Anopheles arabiensis; Anopheles quadriannulatus; Anopheles stephensi; Anopheles dirus, Anopheles arabiensis; Anopheles funestus; and Anopheles melas.

10. The method according to any one of claims 1 to 5, claim 7 or claim 8, wherein the arthropod is a Rhipicephalus spp, optionally wherein the Rhipicephalus spp is Rhipicephalus appendiculatus.

11. The method according to any preceding claim, wherein steps B (a) (i) or B(b) (ii) further comprise:
(aa) centrifuging the homogenate at low speed, of equal to, or less than 140 xg; and
(bb) obtaining parasites retained in the supernatant.

12. The method according to any preceding claim, wherein filtering comprises passing the homogenate sequentially through size exclusion filters.

13. The method according to claim 12, wherein the homogenate is passed through, sequentially:
(aa) a 200 μm to 70 μm pore size filter;
(bb) a 7 μm to 4 μm pore size filter;
(cc) a 4 μm to 25 μm pore size filter;
(dd) a 25 μm to 15 μm pore size filter; and/or
(ee) a 15 μm to 5 μm pore size filter.

14. The method according to either claim 12 or claim 13, wherein the homogenate is passed through, sequentially:
(aa) a 100 μm pore size filter;
(bb) a 70 μm pore size filter
(cc) a 4 μm pore size filter; and
(dd) a 20 μm pore size filter;

15. The method according to any preceding claim, wherein the pre-purifying step is performed by density gradient purification or gel filtration.

16. The method according to claim 15, wherein the pre-purification step comprises:
aa) loading filtered homogenate onto a density gradient;
bb) centrifuging the homogenate present in the density gradient of step aa); and
cc) obtaining parasites from the parasite enriched boundary.

17. The method according to claim 16, wherein the pre-purification step further comprises:
(dd) centrifuging parasites obtained in step (cc);
(ee) aspirating the supernatant resulting from step (dd); and
(ff) resuspending the parasites in buffer.

18. The method according to claim 15, wherein the pre-purification step is performed by cross-linked dextran gel filtration, or G-15 Medium grade gel filtration.

19. The method according to any preceding claim, wherein separation step (C) comprises continuous zone electrophoresis or interval zone electrophoresis.

20. An obligate parasite of vertebrates and arthropods obtained or obtainable by the method of any one of claims 1 to 19.

21. A preparation of attenuated obligate parasite of vertebrates and arthropods, wherein:
i) the preparation is capable of inducing a cellular and humoral immune response when introduced into a subject;
ii) the preparation does not comprise detectable amounts of free circumsporozoite protein (CSP);
iii) the preparation does not comprise detectable amounts of arthropod protein, preferably when assessed by silver stain;
iv) the preparation does not comprise detectable bacterial contamination, preferably when assessed by colony forming units;
v) the preparation enables detection of parasite proteins by mass spectrometry;
vi) the preparation of parasites show no reduction in gliding motility;
vii) the preparation, when introduced into an in vitro culture of hepatocytes, results in infection of at least 1% of total hepatocytes present in the culture;
viii) the preparation, when introduced into an in vitro culture of peripheral blood mononuclear cells, results in infection of at least 1% of total peripheral blood mononuclear cells present in the culture;
ix) the preparation, when injected in vivo into rodents, shows improved time to patency; and/or
x) the preparation, when injected in vivo into bovines, shows improved time to patency.

22. The preparation according to claim 21, wherein the obligate parasite is a parasite selected from the group consisting of: Plasmodium species parasite, Theileria species parasite, Trypanosoma species parasite, Leishmania species parasite and a protozoan parasites of arthropods and vector borne viruses.

23. The preparation according to either claim 21 or claim 22, wherein the obligate parasite is a Plasmodium species parasite, optionally wherein the obligate parasite is P. falciparum.

24. The preparation according to either claim 21 or claim 22, wherein the obligate parasite is a Theileria species parasite, optionally wherein the obligate parasite is T. parva

25. A pharmaceutical composition comprising an attenuated obligate parasite of vertebrates and arthropods according to any one of claims 20 to 24, and a pharmaceutically acceptable vehicle.

26. An attenuated obligate parasite of vertebrates and arthropods according to any one of claims 20 to 24, or the pharmaceutical composition according to claim 25, for use as a medicament.

27. An attenuated obligate parasite of vertebrates and arthropods according to any one of claims 20 to 24, or the pharmaceutical composition according to claim 25, for use in the prevention, amelioration or treatment of a parasitic infection.

28. The attenuated obligate parasite of vertebrates and arthropods according to any one of claims 20 to 23, or the pharmaceutical composition according to claim 25, for use according to claim 27, wherein the attenuated obligate parasite is a Plasmodium spp. and the infection is malaria.

29. The attenuated obligate parasite of vertebrates and arthropods according to any one of claims 20 to 22, claim 24, or the pharmaceutical composition according to claim 25, for use according to claim 27, wherein the attenuated obligate parasite is a Theileria species and the infection is East Coast fever.

30. An attenuated obligate parasite of vertebrates and arthropods according to any one of claims 20 to 24, or the pharmaceutical composition according to claim 25, for use as a vaccine.

31. A vaccine comprising an attenuated obligate parasite of vertebrates and arthropods according to any one of claims 20 to 24, or the pharmaceutical composition according to claim 25.
